(19) United States
(12) Patent Application Publication
(10) Pub. No.: US 2025/0074871 A1
(43) Pub. Date: Mar. 6, 2025

(54) PHENETHYLAMINES AND METHODS OF PREPARATION THEREOF

(71) Applicant: BRIGHT MINDS BIOSCIENCES INC., Vancouver (CA)

(72) Inventors: Alan KOZIKOWSKI, Wallace, MI (US); Werner TUECKMANTEL, Fitchburg, WI (US)

(21) Appl. No.: 18/726,389

(22) PCT Filed: Jan. 4, 2023
(86) PCT No.: PCT/CA2023/050003

§ 371 Date: Jul. 2, 2024

Related U.S. Application Data
(60) Provisional application No. 63/422,730, filed on Nov. 4, 2022.
(60) Provisional application No. 63/296,430, filed on Jan. 4, 2022.

(51) Int. Cl. C07C 323/32; A61K 31/137; A61K 31/277; A61P 9/12; A61P 25/18; C07C 215/52; C07C 217/60; C07C 217/70; C07C 255/59

(52) U.S. Cl. CPC C07C 323/32; A61K 31/137; A61K 31/277; A61P 9/12; A61P 25/18; C07C 215/52; C07C 217/60; C07C 217/70; C07C 255/59; C07C 2601/02

(57) ABSTRACT

The present disclosure relates to compounds of Formula I that exhibit 5-HT_{2A} receptor agonist activity and low 5HT_{2B} receptor agonist activity or deemed 5HT_{2B} receptor agonist inactivity. In at least some cases, such compounds show selectivity for the 5-HT_{2A} receptor over the 5-HT_{2C} receptor. As contemplated herein, phenethylamines may be used for the treatment of neuropsychiatric, neurodegenerative, neuroinflammatory and pain disorders including depression, drug (e.g. tobacco, opiate, and cocaine) addiction, alcoholism, post-traumatic stress disorder (PTSD), and neuropathic pain syndromes including cluster headaches and chemotherapy induced peripheral neuropathy.

### TECHNICAL FIELD

This present disclosure relates to phenethylamines and methods of preparing the same.

The present disclosure also relates to uses of phenethylamines as selective agents at serotonin receptors.

### BACKGROUND

Psilocybin is a naturally occurring psychedelic compound produced by more than 200 species of mushrooms collectively known as “psilocybin mushrooms”. As a prodrug, psilocybin is quickly metabolized by the body to generate the bioactive compound psilocin, which has mind-altering effects not unlike those produced by other psychedelics such as lysergic acid diethylamide (LSD), mescaline, and N,N-dimethyltryptamine (DMT). These effects include, inter alia, euphoria, visual and mental hallucinations, changes in perception, distortions in one's sense of time, and spiritual experiences; these effects can also include possible adverse reactions such as nausea and panic attacks. For reference, the chemical structure of psilocin is provided in FIG. 1 herein.

As agonists of the 5-HT_{2A} and 5-HT_{2C} receptors, psilocybin and psilocin have been recognized for their therapeutic potential. Since 5-HT_{2A} receptor activation appears to increase locomotor activity whereas 5-HT_{2C} receptor activation appears to decrease locomotor activity, compounds possessing varying degrees of 5-HT_{2A} and 5-HT_{2C} activity will show varying levels of psychedelic activity (Halberstadt A L, van der Heijden I, Ruderman M A, Risbrough V B, Gingrich J A. Geyer M A, Powell S B, Neuropsychopharmacology, 2009, 34(8):1958-67). While psilocybin, along with other psychedelic drugs, were explored more than 60 years ago by Hofmann and his co-workers at the time (see for example, Hofmann, A., Troxler, F. U.S. Pat. Nos. 3,075,992; 3,078,214), clinical investigations into these drugs waned substantially by the early 1970s—particularly after these drugs were placed on Schedule 1 of the Controlled Substance Act in the United States of America. Notwithstanding their listing as controlled substances in certain jurisdictions however, research into psilocybin and other psychedelic drugs never fully stopped, and recent clinical investigations have led to a revived interest in the potential application of psychedelic drugs (including psilocybin) in evolving medical areas, such as the treatment of central nervous system (CNS) diseases. CNS diseases include both difficult-to-treat mental health disorders (Daniel J, Haberman M. Clinical potential of psilocybin as a treatment for mental health conditions. Ment. Health Clin. 2017, 7(1), 24-8), such as treatment resistant depression or drug resistant depression, and neurological disorders such as cluster headaches.

While psilocybin has recognized therapeutic potential for treating certain CNS diseases and disorders, it is also recognized as a 5-HT_{2B} receptor agonist which means that psilocybin exhibits cardiotoxic activity. As such, there is an unmet need for safer drugs that at least lack cardiotoxic 5-HT_{2B} agonist activity. Furthermore, and at least in some instances, there is an unmet need for safer drugs that maintain 5-HT_{2A} receptor agonist activity but that lack cardiotoxic 5-HT_{2B} agonist activity.

In recent years, a variety of N-substituted phenethylamines have been identified as 5-HT_{2A} selective. Notably, 25CN-NBOH and closely related analogs have been found to show selectivity for the 5-HT_{2A} receptor over the 5-HT_{2C} receptor and thus exhibit more robust psychedelic activity (see Synthesis and structure-activity relationships of N-benzyl phenethylamines as 5-HT_{2A/2C} agonists. Hansen M, Phonekeo K, Paine J S, Leth-Petersen S, Begtrup M, Brauner-Osborne H, Kristensen J L. ACS Chem Neurosci. 2014 Mar. 19; 5(3): 243-9. doi: 10.1021/cn400216u). However, these compounds are not without their potential drawbacks. Firstly, certain members of this phenethylamine class of compounds may generate reactive benzoquinone metabolites, though it has been found that removal of one of the methoxy groups from such compounds can mitigate against the generation of such metabolites while retaining 5-HT_{2A} selectivity. Secondly, certain members of this phenethylamine class of compounds have been shown to be extremely toxic to humans, notably causing cardiac arrest (Zawilska J B, Kacela M, Adamowicz P, Front. Neurosci. 2020; 14: 78).

### SUMMARY

The present disclosure relates to compounds that belong to the phenethylamine class of molecules that exhibit 5-HT_{2A} receptor agonist activity while exhibiting low 5HT_{2B} receptor agonist activity or deemed 5HT_{2B} receptor agonist inactivity. In at least some cases, such compounds show selectivity for the 5-HT_{2A} receptor over the 5-HT_{2C} receptor. The compounds disclosed herein may be useful in the treatment of depression including major depressive disorder, drug resistant depression, and psychotic depression, addiction including alcoholism, tobacco addiction, cocaine addiction, and opioid addiction, pain indications including neuropathic pain, pain from chemotherapy associated neuropathy, phantom limb pain and fibromyalgia, inflammation (including chronic and acute), eating disorders including anorexia, autism, cluster headaches, migraines, dementia including Alzheimer's dementia, Parkinson's disease dementia and Lewy body dementia, post-traumatic stress disorder, emotional distress associated with cancer, Fragile-X syndrome, autism spectrum disorder, bipolar disease, obsessive compulsive disease, Rett syndrome, and other CNS disorders.

According to a part of the present disclosure, there are chemical entities of Formula I, or isotopologues or pharmaceutically acceptable salts thereof:

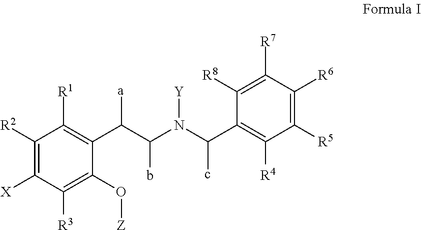

wherein R¹, R², R³, R⁴, R⁵, R⁶, R⁷, R⁸, X, Y, Z, a, b, and c are defined herein.

According to another part of the present disclosure, there are chemical entities of Formula I, or isotopologues or pharmaceutically acceptable salts thereof:

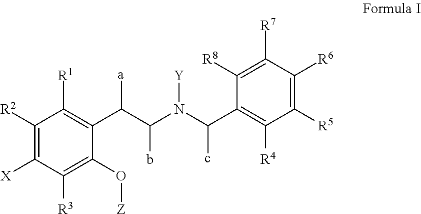

wherein: (a) R¹: (i) may be selected from the group consisting of H, C₁-C₄ alkyl, substituted C₁-C₄ alkyl, cyclopropyl, cyclobutyl, cyclopropylmethyl, 2-oxetanyl, 3-oxetanyl, OH, C₁-C₄ alkoxy, substituted C₁-C₄ alkoxy, C₁-C₄ alkylthio, substituted C₁-C₄ alkylthio, and halogen, if each of R² and R³ is independently selected from H, CH₃, or halogen; or (ii) together with R² form an alkanediyl, alkenediyl, heteroalkanediyl, or heteroalkenediyl moiety; or (iii) together with b form an alkanediyl, alkenediyl, or heteroalkanediyl moiety; (b) R²: (i) may be selected from the group consisting of H, C₁-C₄ alkyl, substituted C₁-C₄ alkyl, cyclopropyl, cyclobutyl, cyclopropylmethyl, 2-oxetanyl, 3-oxetanyl, OH, C₁-C₄ alkoxy, substituted C₁-C₄ alkoxy, C₁-C₄ alkylthio, substituted C₁-C₄ alkylthio, and halogen, if each of R¹ and R³ is independently selected from H, CH₃, or halogen; or (ii) together with R¹ form an alkanediyl, alkenediyl, heteroalkanediyl, or heteroalkenediyl moiety; and (c) R³: (i) may be selected from the group consisting of H, C₁-C₄ alkyl, substituted C₁-C₄ alkyl, cyclopropyl, cyclobutyl, cyclopropylmethyl, 2-oxetanyl, 3-oxetanyl, OH, C₁-C₄ alkoxy, substituted C₁-C₄ alkoxy, C₁-C₄ alkylthio, substituted C₁-C₄ alkylthio, and halogen, if each of R¹ and R² is independently selected from H, CH₃, or halogen; or (ii) together with Z form an alkanediyl, alkenediyl, heteroalkanediyl, or heteroalkenediyl moiety.

One of R⁴, R⁵, R⁶, R⁷, and R³ may be selected from the group consisting of R⁹, OR⁹, SR⁹, S(O)R⁹, S(O)₂R⁹, N(R⁹)C(O)R⁹, N(R⁹)C(O)OR⁹, N(R⁹)C(O)N(H)R⁹, N(R⁹)C(O)N(C₁-C₆ alkyl)R⁹, N(R⁹)S(O)₂R⁹, CH₂OR⁹, CH₂SR⁹, CH₂S(O)R⁹, CH₂S(O)₂R⁹, CH₂N(R⁹)C(O)R⁹, CH₂N(R⁹)C(O)OR⁹, CH₂N(R⁹)C(O)N(H)R⁹, CH₂N(R⁹)C(O)N(C₁-C₆ alkyl)R⁹, CH₂N(R⁹)S(O)₂R⁹, CH═NOR⁹, C(C₁-C₆ alkyl)=NOR⁹, C(O)OR⁹, C(O)N(H)R⁹, C(O)N(C₁-C₆ alkyl)R⁹, CH₂C(O)OR⁹, CH₂C(O)N(H)R⁹, CH₂C(O)N(C₁-C₆ alkyl)R⁹, CN, and halogen, a second one of R⁴, R⁵, R⁶, R⁷ or R³ may be selected from the group consisting of H, C₁-C₄ alkyl, CHF₂, CF₃, OH, OCH₃, OC₂H₅, OCHF₂, OCF₃, and halogen, and the remainder of R⁴, R⁵, R⁶, R⁷, and R³ may be each independently H, CH₃, or halogen. If R⁴ and R⁵ together form an alkanediyl, alkenediyl, heteroalkanediyl, or heteroalkenediyl moiety, then each of R⁶, R⁷, and R³ may be independently selected from the group consisting of H, CH₃, and halogen. If R⁴ and b together form an alkanediyl, alkenediyl, or heteroalkanediyl moiety, then one of R⁵, R⁶, R⁷, and R³ may be selected from the group consisting of H, C₁-C₄ alkyl, CHF₂, CF₃, OH, OCH₃, OC₂H₅, OCHF₂, OCF₃, and halogen, and the remainder of R⁵, R⁶, R⁷, and R³ may each be independently selected from the group consisting of H, CH₃, and halogen. If R⁴ and c together form an alkanediyl, alkenediyl, or heteroalkanediyl moiety, then one of R⁵, R⁶, R⁷, and R³ may be selected from the group consisting of H, C₁-C₄ alkyl, CHF₂, CF₃, OH, OCH₃, OC₂H₅, OCHF₂, OCF₃, CN, and halogen, and the remainder of R⁵, R⁶, R⁷, and R³ may each be independently selected from the group consisting of H, CH₃, and halogen.

X may be selected from the group consisting of CN, C(O)NH₂, C(O)N(H)R⁹, C(O)N(C₁-C₆ alkyl)R⁹, C(O)(C₄-C₆ heterocyclyl), CHF₂, CF₃, OH, O(C₁-C₆ alkyl), OCHF₂, OCF₃, S(C₁-C₆ alkyl), SCF₃, SCHF₂, F, C, aryl, and heteroaryl generally. Y may be selected from the group consisting of H, CH₃, C(O)R⁹, CH₂OC(O)R⁹, and C(O)OR⁹. Z: (i) may be selected from the group consisting of H, C₁-C₁₀ alkyl, substituted C₁-C₁₀ alkyl, C₁-C₁₀ heteroalkyl, C₂-C₁₀ alkenyl, C₂-C₁₀ heteroalkenyl, C₂-C₁₀ alkynyl, C₃-C₆ cycloalkyl, (C₃-C₆ cycloalkyl)(C₁—C alkyl), (C₃-C₆ cycloalkyl)(C₁-C₈ heteroalkyl), C₄-C₆ heterocyclyl, (C₄-C₆ heterocyclyl)(C₁-C₃ alkyl), (C₄-C₆ heterocyclyl)(C₁-C₃ heteroalkyl), aryl(C₁-C₈ alkyl), aryl(C₁-C₃ heteroalkyl), heteroaryl(C₁—C alkyl), heteroaryl(C₁-C₃ heteroalkyl), C(O)R⁹, C(O)OR⁹, CH₂OC(O)R⁹, C(O)NH₂, C(O)N(H)R⁹, C(O)N(C₁-C₆ alkyl)R⁹, and C(O)(C₄-C₆ heterocyclyl); or (ii) together with R³ may form an alkanediyl, alkenediyl, heteroalkanediyl, or heteroalkenediyl moiety.

Two of a, b, and c may each be independently selected from the group consisting of H, CH₃, and C₂H₅, while the third of a, b, and c may be H, but if c and R⁴ together form an alkanediyl, alkenediyl, or heteroalkanediyl moiety then a and b may each be independently selected from H or CH₃.

R⁹ may, independently for each occurrence, be selected from the group consisting of H, C₁-C₆ alkyl, C₁-C₆ heteroalkyl, substituted C₁-C₆ alkyl, substituted C₁-C₆ heteroalkyl, C₂-C₆ alkenyl, C₂-C₆ heteroalkenyl, C₂-C₆ alkynyl, C₃-C₆ cycloalkyl, (C₃-C₆ cycloalkyl)(C₁-C₆ alkyl), (C₃-C₆ cycloalkyl)(C₁-C₆ heteroalkyl), C₃-C₆ heterocyclyl, (C₃-C₆ heterocyclyl)(C₁-C₆ alkyl), (C₃-C₆ heterocyclyl)(C₁-C₆ heteroalkyl), aryl, aryl(C₁-C₆ alkyl), aryl(C₁-C₆ heteroalkyl), heteroaryl, heteroaryl(C₁-C₆ alkyl), and heteroaryl(C₁-C₆ heteroalkyl).

The chemical entities of Formula I disclosed herein are 5-HT_{2A} subtype selective receptor agonists. Chemical entities of Formula I, and isotopologues and pharmaceutically acceptable compositions thereof, are believed to be useful for treating a variety of diseases and disorders associated with 5-HT_{2A} receptor agonism. Such diseases and disorders include those described herein.

This summary does not necessarily describe the entire scope of all aspects of the disclosure. Other aspects, features and advantages will be apparent to those of ordinary skill in the art upon review of the following description of specific embodiments.

### BRIEF DESCRIPTION OF DRAWINGS

In the accompanying drawings, which illustrate one or more embodiments:

FIG. 1 depicts the chemical structure of psilocin.

FIG. 2 depicts the chemical structure of Formula I.

FIG. 3 is a graph depicting an HTR versus dose for compound 109 (as described herein), as administered to mice.

FIG. 4 is a graph depicting an HTR over time per dose (mg/kg) for compound 109 (as described herein), as administered to mice.

FIG. 5 is a graph depicting the horizontal activity and vertical activity of rat subjects in locomotor activity tests, in response to a intraperitoneal dose of compound 109 (as described herein).

FIG. 6 depicts a synthetic scheme for compounds having a chemical structure of Formula I.

FIG. 7 depicts a synthetic scheme of a benzyl moiety in compounds having a chemical structure of Formula I.

FIG. 8 depicts a synthetic scheme of a phenethyl moiety in compounds having a chemical structure of Formula I.

FIG. 9a depicts a synthetic scheme of compound 12 (as described herein).

FIG. 9b depicts another synthetic scheme of compound 12 (as described herein).

FIG. 10 depicts a synthetic scheme of a phenethylamine building block for compound 32 (as described herein).

FIG. 11 depicts a synthetic scheme for a benzylamine building block for compound 76 (as described herein).

FIG. 12a depicts a moiety of a chemical structure of Formula I, wherein a is a methyl group.

FIG. 12b depicts a moiety of a chemical structure of Formula I, wherein b is a methyl group.

FIG. 12c depicts a moiety of a chemical structure of Formula I, wherein c is a methyl group.

FIG. 13 depicts a scheme for the synthesis of one of four stereoisomers of compound 86 (as described herein), in which b and c are connected to form a ring.

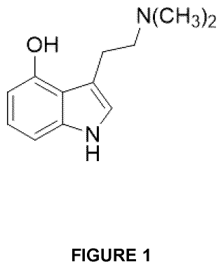

### DETAILED DESCRIPTION

Directional terms such as “top,” “bottom,” “upwards,” “downwards,” “vertically,” and “laterally” are used in the following description for the purpose of providing relative reference only, and are not intended to suggest any limitations on how any article is to be positioned during use, or to be mounted in an assembly or relative to an environment. The use of the word “a” or “an” when used herein in conjunction with the term “comprising” may mean “one,” but it is also consistent with the meaning of “one or more,” “at least one” and “one or more than one.” Any element expressed in the singular form also encompasses its plural form. Any element expressed in the plural form also encompasses its singular form. The term “plurality” as used herein means more than one, for example, two or more, three or more, four or more, and the like.

As used herein and unless otherwise specified, the term “about”, when used to describe a recited value, means within 10% of the recited value.

As used herein and unless otherwise specified, the term “alkanediyl”, used alone or as part of a larger moiety, means a substituted or unsubstituted, linear, divalent hydrocarbon chain that can be attached by way of its terminal carbon atoms and that is completely saturated. Unless otherwise specified, an alkanediyl group contains 1 to 5 carbon atoms (“C₁-C₅ alkanediyl”). Non-limiting examples of alkanediyl groups include methylene, ethylene, trimethylene, tetramethylene, and pentamethylene. A substituted alkanediyl group is one having at least one but no more than five substituents, and no more substituents than the number of hydrogen atoms in the unsubstituted group. Non-limiting examples of substituted alkanediyl groups include difluoromethylene, hydroxyethylene, methoxyethylene, CH₂CH₂C(O), CH₂C(CH₃)₂CH₂, CH₂CH₂CH(CF₃)CH₂, and the like.

As used herein and unless otherwise specified, the term “alkenediyl”, used alone or as part of a larger moiety, means a substituted or unsubstituted, linear, divalent hydrocarbon chain having at least two carbon atoms and at least one carbon-carbon double bond, that can be attached by way of its terminal carbon atoms. Unless otherwise specified, an alkenediyl group contains 2 to 5 carbon atoms (“C₂-C₅ alkenediyl”). Non-limiting examples of alkenediyl groups include vinylene (CH═CH), propene-1,3-diyl(CH₂CH═CH), 1-butene-1,4-diyl(CH₂CH₂CH═CH), 2-butene-1,4-diyl(CH₂CH═CHCH₂), and 1,3-butadiene-1,4-diyl(CH═CHCH═CH). For illustration purposes, if a first end of a 1,3-butadiene-1,4-diyl group is attached to a first carbon atom of a carbon-carbon (CC) double bond or an aromatic ring, and a second end of the 1,3-butadiene-1,4-diyl group is attached to a second carbon atom of the CC double bond or the aromatic ring, and the second carbon atom is adjacent to the first carbon atom, then said two adjacent carbon atoms and the 1,3-butadiene-1,4-diyl group together represent a benzene ring. For illustration purposes, if a first end of a vinylene group is attached to a first carbon atom of a CC double bond or an aromatic ring, and a second end of the vinylene group is attached to a heteroatom (selected from the group consisting of N, O, and S) that is adjacent to a second carbon atom of the CC double bond or the aromatic ring, and the second carbon atom is adjacent to the first carbon atom, then the vinylene group together with said two adjacent carbon atoms and said heteroatom attached to the second carbon atom represent a pyrrole, furan, or thiophene ring.

As used herein and unless otherwise specified, the term “alkenyl” refers to a substituted or unsubstituted, linear or branched, univalent hydrocarbon chain having at least two carbon atoms and at least one carbon-carbon double bond. Examples of alkenyl groups include allyl, vinyl, 2-propenyl, crotyl, 2-isopentenyl, 1,3-butadien-2-yl, 2,4-pentadien-1-yl, 1,4-pentadien-3-yl, and the like.

As used herein and unless otherwise specified, the term “alkoxy”, used alone or as part of a larger moiety, refers to the groups —O-alkyl and —O-cycloalkyl. As used herein and unless otherwise specified, the term “substituted alkoxy”, used alone or as part of a larger moiety, refers to the groups -O-(substituted alkyl) and -O-(substituted cycloalkyl).

As used herein and unless otherwise specified, the term “alkyl”, used alone or as part of a larger moiety, means a substituted or unsubstituted, linear or branched, univalent hydrocarbon chain that is completely saturated. Unless otherwise specified, an alkyl group contains 1 to 7 carbon atoms (“C₁-C₇ alkyl”). For example, in some embodiments, alkyl groups contain 1 to 6 carbon atoms (“C₁-C₆ alkyl”); in some embodiments, alkyl groups contain 1 to 5 carbon atoms (“C₁-C₅ alkyl”); in some embodiments, alkyl groups contain 1 to 4 carbon atoms (“C₁-C₄ alkyl”, alternatively “lower alkyl”); and in some embodiments, alkyl groups contain 3 to 7 carbon atoms (“C₃-C₇ alkyl”). Non-limiting examples of saturated alkyl groups include methyl, ethyl, n-propyl, i-propyl, n-butyl, t-butyl, i-butyl, s-butyl, homologs and isomers of, for example, n-pentyl, n-hexyl, n-heptyl, n-octyl, and the like. Examples of lower alkyl groups include methyl, ethyl, n-propyl, i-propyl, n-butyl, s-butyl, i-butyl, and t-butyl. A substituted alkyl group is one having at least one but no more than five substituents, and no more substituents than the number of hydrogen atoms in the unsubstituted group. In some embodiments, the substituents are fluorine atoms. Non-limiting examples of substituted alkyl groups include 2-hydroxyethyl, 2-methoxyethyl, CHF₂, CF₃, CH₂CF₃, CF₂CF₃, 4-fluorobutyl, and the like.

As used herein and unless otherwise specified, the term “alkylthio”, used alone or as part of a larger moiety, refers to the groups —S-alkyl and —S-cycloalkyl. “Substituted alkylthio”, used alone or as part of a larger moiety, refers to the groups -S-(substituted alkyl) and -S-(substituted cycloalkyl).

As used herein and unless otherwise specified, the term “alkynyl” refers to a substituted or unsubstituted, linear or branched, univalent hydrocarbon chain having at least two carbon atoms and at least one carbon-carbon triple bond. Non-limiting examples of alkynyl groups include ethynyl, 1—and 3-propynyl, 3-butyn-1-yl, and the like.

As used herein and unless otherwise specified, the term “aryl”, used alone or as part of a larger moiety (for example, “(aryl)alkyl”) refers to a univalent monocyclic or bicyclic carbocyclic aromatic ring system. Unless otherwise specified, aryl groups contain 6 or 10 ring members. Non-limiting examples of aryl include phenyl, naphthyl, and the like. The term “aryl” also refers to aryl groups that may be unsubstituted or substituted. For example, aryl groups can be unsubstituted or can be substituted with one, two, or three groups selected independently from the group consisting of halogen, OH, C₁-C₆ alkoxy, substituted C₁-C₆ alkoxy, C₁-C₆ alkylthio, substituted C₁-C₆ alkylthio, C₁-C₆ alkyl, substituted C₁-C₆ alkyl, C₃-C₆ cycloalkyl, substituted C₃-C₆ cycloalkyl, C(O)OH, C(O)(C₁-C₆ alkyl), C(N-OH)(C₁-C₆ alkyl), C(O)(C₁-C₆ alkoxy), C(O)NH₂, C(O)NH(C₁-C₆ alkyl), C(O)N(C₁-C₄ alkyl)(C₁-C₄ alkyl), C(O)-heterocyclyl, NHC(O)(C₁-C₆ alkyl), N(CH₃)C(O)(C₁-C₆ alkyl), and cyano.

As used herein and unless otherwise specified, the term “chemical entity” refers to a compound having the indicated structure, whether in its “free” form (e.g., “free compound” or “free base” or “free acid” form, as applicable), or in a salt form, particularly a pharmaceutically acceptable salt form, and furthermore whether in solid state form or otherwise. In some embodiments, a solid state form is an amorphous (i.e., non-crystalline) form; in some embodiments, a solid state form is a crystalline form (e.g., a polymorph, pseudohydrate, hydrate, or solvate). Similarly, the term encompasses the compound whether provided in solid form or otherwise. Unless otherwise specified, all statements made herein regarding “compounds” apply to the associated chemical entities, as defined.

As used herein and unless otherwise specified, the terms “comprising”, “having”, “including”, “containing”, and grammatical variations thereof, are inclusive or open-ended and do not exclude additional, un-recited elements and/or method steps. For example, “A includes 1, 2, and 3” means that A includes but is not limited to 1, 2, and 3.

As used herein and unless otherwise specified, the term “consisting essentially of” when used herein in connection with a composition, use, or method, denotes that additional elements, method steps or both additional elements and method steps may be present, but that these additions do not materially affect the manner in which the recited composition, method, or use functions.

As used herein and unless otherwise specified, the term “consisting of” when used herein in connection with a composition, use, or method, excludes the presence of additional elements and/or method steps.

As used herein, the term “cpd” is an abbreviation for the word “compound”.

As used herein and unless otherwise specified, the term “cycloalkyl”, used alone or as part of a larger moiety, for example “(cycloalkyl)alkyl”, refers to: (i) a substituted or unsubstituted, univalent monocyclic hydrocarbon radical that is completely saturated or that contains one or more units of unsaturation, but which is not aromatic; or (ii) bicyclo[m.n.o]alkyl wherein each of “m”, “n”, and “o” is independently an integer ranging from zero to 5, and the sum “m”+“n”+“o” ranges from 2 to 6. In some embodiments, cycloalkyl groups contain 3 to 8 ring carbon atoms (“C₃-C₈ cycloalkyl”). Non-limiting examples of cycloalkyl include cyclopropyl, cyclobutyl, cyclopentyl, cyclohexyl, 1-cyclohexenyl, 3-cyclohexenyl, cycloheptyl, and the like, as well as bicyclo[2.2.1]heptyl (also called norbornyl) and bicyclo[1.1.1]pentyl. A substituted cycloalkyl group is one having at least one but no more than five substituents. In some embodiments, the substituents are fluorine atoms. Non-limiting examples of substituted cycloalkyl groups include 2-methylcyclopropyl, 4-hydroxycyclohexyl, 2-methoxycyclopentyl, 4,4-difluorocyclohexyl, and the like.

As used herein and unless otherwise specified, the term “halogen” or “halo”, used alone or as part of a larger moiety, refers to fluoro, chloro, bromo, or iodo.

As used herein and unless otherwise specified, the term “hERG risk” refers to the risk of a compound potentially inhibiting the human ether-a-go-go-related gene (hERG) K⁺ channel, thereby leading to QT prolongation.

As used herein and unless otherwise specified, the term “heteroalkanediyl” refers to a substituted or unsubstituted alkanediyl group, as defined herein, in which one or more of the constituent carbon atoms have been replaced by nitrogen, oxygen, or sulfur. Non-limiting examples for heteroalkanediyl groups include NH, O, S, CH₂NH, CH₂O, CH₂S, CH₂CH₂NH, CH₂CH₂O, CH₂CH₂S, CH₂NHCH₂, CH₂OCH₂, CH₂SCH₂, OCH₂O, OCH₂S, SCH₂S, CH₂CH₂CH₂NH, CH₂CH₂NHCH₂, CH₂CH₂CH₂O, CH₂CH₂OCH₂, CH₂CH₂CH₂S, and CH₂CH₂SCH₂. For illustration purposes, if the points of attachment of a heteroalkanediyl group are non-equivalent (e.g., CH₂CH₂NHCH₂), and the points are attached to non-equivalent positions of a chemical structure, then the attachment of the heteroalkanediyl group to the chemical structure can occur in either direction (e.g., CH₂CH₂NHCH₂ or CH₂NHCH₂CH₂).

As used herein and unless otherwise specified, the term “heteroalkenediyl” refers to a substituted or unsubstituted alkenediyl group, as defined herein, in which one or more of the constituent carbon atoms have been replaced by nitrogen, oxygen, or sulfur. Non-limiting examples for heteroalkenediyl groups include CH═N, CH═CHNH, CH═CHO, CH═CHS, CH═NCH₂, N═N, CH═NNH, CH═NO, CH═NS, NHCH═N, OCH═N, SCH═N, HNN═N, CH═CHCH=N, CH═CHN═CH, CH═CHN═N, CH═NCH=N, N═CHCH═N, CH═NN═CH, CH═CHCH₂NH, CH═CHCH₂O, and CH═CHCH₂S. For illustration purposes, if the points of attachment of a heteroalkenediyl group are non-equivalent (e.g., in CH═CHS), and the points are attached to non-equivalent positions of a chemical structure, then the attachment of the heteroalkenediyl group to the chemical structure can occur in either direction (e.g., CH═CHS or SCH═CH). For illustration purposes, if a first point of attachment of a heteroalkenediyl group is attached to a first carbon atom of a CC double bond or an aromatic ring, and a second point of attachment of the heteroalkenediyl group is attached to a second carbon atom of the CC double bond or the aromatic ring, and the first carbon atom and the second carbon atom are adjacent to each other, then said two adjacent carbon atoms and the heteroalkenediyl group together represent a heteroaromatic ring; particularly, (i) if the formed heteroaromatic ring is five-membered, then the single saturated atom of the heteroalkenediyl group is selected from the group consisting of N, O, and S, and (ii) if the formed heteroaromatic ring is a six-membered, then the heteroalkenediyl group comprises only C and N atoms as a part of its backbone and contains two conjugated double bonds. For illustration purposes, if a first point of attachment of a heteroalkenediyl group represented by the formula X=Y, wherein X and Y are independently selected from the group consisting of C and N, is attached to a first carbon atom of a CC double bond or an aromatic ring, and a second point of attachment of the heteroalkenediyl group is attached to a heteroatom (selected from the group consisting of N, O, and S) that in turn is attached to a second carbon atom of the CC double bond or the aromatic ring, and the second carbon atom is adjacent to the first carbon atom, then the moiety X=Y together with said two adjacent carbon atoms and said heteroatom attached to one of them represent a five-membered heteroaromatic ring.

As used herein and unless otherwise specified, the term “heteroalkenyl” refers to a substituted or unsubstituted alkenyl group, as defined herein, in which one or more of the constituent carbon atoms have been replaced by nitrogen, oxygen, or sulfur.

As used herein and unless otherwise specified, the term “heteroalkyl” refers to a substituted or unsubstituted alkyl group, as defined herein, in which one or more of the constituent carbon atoms have been replaced by nitrogen, oxygen, or sulfur.

As used herein and unless otherwise specified, the term “heteroaryl”, used alone or as part of a larger moiety, e.g., “(heteroaryl)alkyl”, refers to a univalent monocyclic or bicyclic group having 5 to 10 ring atoms, preferably 5, 6, 9, or 10 ring atoms, having 6 or 10 rr electrons shared in a cyclic array, and having, in addition to ring carbon atoms, from one to four ring heteroatoms. Examples of heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolyl, indolizinyl, benzofuranyl, benzothiophenyl, benzimidazolyl, benzoxazolyl, benzothiazolyl, benzotriazolyl, quinolyl, isoquinolyl, purinyl, naphthyridinyl, pteridinyl, and the like.

Heteroaryl groups may be unsubstituted or may be substituted with one, two, or three groups selected independently from halogen, OH, C₁-C₆ alkoxy, substituted C₁-C₆ alkoxy, C₁-C₆ alkylthio, substituted C₁-C₆ alkylthio, C₁-C₆ alkyl, substituted C₁-C₆ alkyl, C₃-C₆ cycloalkyl, substituted C₃-C₆ cycloalkyl, C(O)OH, C(O)(C₁-C₆ alkoxy), C(O)NH₂, C(O)NH(C₁-C₆ alkyl), C(O)N(C₁-C₄ alkyl)(C₁-C₄ alkyl), C(O)-heterocyclyl, NHC(O)(C₁-C₆ alkyl), N(CH₃)C(O)(C₁-C₆ alkyl), and cyano.

As used herein and unless otherwise specified, the term “heterocyclyl”, used alone or as part of a larger moiety (for example, “(heterocyclyl)alkyl”) refers to a univalent stable 4—to 7-membered monocyclic or 7—to 10-membered bicyclic heterocyclic moiety that is either saturated or partially unsaturated, and has, in addition to ring carbon atoms, one to four heteroatoms. Non-limiting examples of heterocyclyl groups include tetrahydrofuranyl, pyrrolidinyl, tetrahydropyranyl, piperidinyl, morpholinyl, and the like. Heterocyclyl groups can be unsubstituted or can be substituted. For example, heterocyclyl groups can be unsubstituted or can be substituted with one, two, or three groups selected independently from the group consisting of halogen, OH, O(C₁-C₆ alkyl), O(substituted C₁-C₆ alkyl), C₁-C₆ alkyl, substituted C₁-C₆ alkyl, and C₃-C₆ cycloalkyl.

As used herein and unless otherwise specified, the term “HTR” refers to head-twitch response, which is a measurement of 5-HT_{2A} activation in vivo.

As used herein and unless otherwise specified, the term “inactive” (and all related terms thereto including “inactivity”), when used the context of “EC₅₀ (nM)” and “Eff %” as such terms would be understood by a person skilled in the art or equivalent skilled person, and when used in reference to the activity at the 5-HT_{2B} receptor, means a concentration of greater than 10,000 nM (when used in the context of “EC₅₀ (nM)”) or an efficacy of 30% or lower (when used in the context of “Eff %”).

As used herein and unless otherwise specified, the term “isotopologue” refers to a species that differs from a specific compound only in the isotopic composition thereof. For example, all hydrogen atoms in a compound are independently of natural isotopic composition or of any isotopic composition enriched or depleted in one or both of the heavy isotopes, ²H (D, deuterium) and ³H (T, tritium), ranging from a depletion to zero % to an enrichment to 100%.

As used herein and unless otherwise specified, the term “low 2B activity” refers to a situation where the 5-HT_{2A} agonist activity (or 5-HT_{2A} receptor sensitivity) of a compound, as measured in terms of “EC₅₀ (nm)”, is 500 times or more greater than the 5-HT_{2B} agonist activity (or 5-HT_{2B} receptor sensitivity) of said compound and the Eff % at each receptor is less than 90% at the measured EC₅₀ for each receptor. For example, a compound is deemed to have “low 2B activity” if such compound possesses: (i) an EC₅₀ and Eff % of 1.0 nm and 95% respectively at the 5-HT_{2A} receptor; and (ii) an EC₅₀ and Eff % of 700 nm and 95% respectively at the 5-HT_{2B} receptor.

As used herein and unless otherwise specified, the term “mice” refers to male C57BL/6J mice (6-8 weeks old).

As used herein and unless otherwise specified, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts of the compounds provided in this disclosure include salts derived from suitable inorganic and organic acids and bases. Non-limiting examples of pharmaceutically acceptable salts include salts of compounds comprising an amino group that are formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid. Other non-limiting examples of pharmaceutically acceptable salts include adipate, alginate, ascorbate, aspartate, benzenesulfonate, benzoate, bisulfate, borate, butyrate, camphorate, camphorsulfonate, cyclopentanepropionate, digluconate, dodecylsulfate, ethanesulfonate, formate, fumarate, glucoheptonate, glycerophosphate, gluconate, hemisulfate, heptanoate, hexanoate, hydriodide, 2-hydroxyethanesulfonate, lactobionate, lactate, laurate, lauryl sulfate, malate, methanesulfonate, 2-naphthalenesulfonate, nicotinate, nitrate, oleate, palmitate, pamoate, pectinate, persulfate, 3-phenylpropionate, pivalate, propionate, stearate, tartrate, thiocyanate, p-toluenesulfonate, undecanoate, valerate salts, and the like. Other pharmaceutically acceptable salts include those that are derived from appropriate bases such as alkali metal, alkaline earth metal, ammonium, and N+(C₁₋₄ alkyl)₄ salts. Representative alkali or alkaline earth metal salts include sodium, lithium, potassium, calcium, magnesium, and the like.

Further non-limiting examples of pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein and unless otherwise specified, the term “subject” includes a mammal (e.g., a human, and in some embodiments including prenatal human forms). In some embodiments, a subject suffers from a relevant disease, disorder, or condition. In some embodiments, a subject is susceptible to a disease, disorder, or condition. In some embodiments, a subject displays one or more symptoms or characteristics of a disease, disorder, or condition. In some embodiments, a subject does not display any symptom or characteristic of a disease, disorder, or condition. In some embodiments, a subject is a mammal with one or more features characteristic of susceptibility to or risk of a disease, disorder, or condition. In some embodiments, a subject is a patient. In some embodiments, a subject is an individual to whom diagnosis and/or therapy is and/or has been administered. In some embodiments, a subject is a fetus, an infant, a child, a teenager, an adult, or a senior citizen (i.e., the subject is of advanced age, such as older than 50). In some embodiments, a child refers to a human that is between two and 18 years of age. In some embodiments, an adult refers to a human that is eighteen years of age or older.

As used herein and unless otherwise specified, the phrase “such as” is intended to be open-ended. For example, the phrase “A can be a halogen, such as chlorine or bromine” means that “A” can be, but is not limited to, chlorine or bromine.

Reference to specific moieties, functional groups, or substituents contemplates (where applicable) tautomers thereof.

Unless otherwise stated, structures depicted herein include all isomeric (e.g., enantiomeric, diastereomeric, and geometric (or conformational)) forms of the structure (e.g., the R and S configurations for each asymmetric center, Z and E double bond isomers, and Z and E conformational isomers). Unless otherwise stated, the compounds disclosed, taught, or otherwise suggested in this disclosure contemplate all single stereochemical isomers as well as enantiomeric, diastereomeric, and geometric (or conformational) mixtures thereof. Unless otherwise stated, the compounds disclosed, taught, or suggested in this disclosure contemplate all tautomeric forms thereof. Additionally, unless otherwise stated, structures depicted herein include compounds that differ only in the presence of one or more isotopically enriched atoms. Such compounds may be useful, for example, as analytical tools, as probes in biological assays, or as therapeutic agents. Additionally, incorporation of heavier isotopes such as deuterium (²H) may afford certain therapeutic advantages resulting from greater metabolic stability, for example, increase in vivo half-life, or reduced dosage requirements.

Chemical entities described herein are further illustrated by the classes, subclasses, and species disclosed herein. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. In this disclosure, any atom not specifically designated as a particular isotope is meant to represent any stable isotope of that atom.

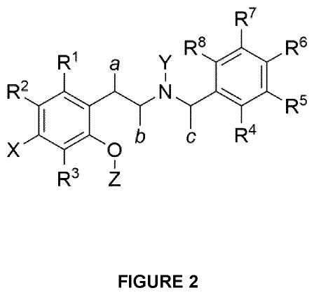

Referring to FIG. 2, and according to embodiments of the chemical entities disclosed herein (including isotopologues or pharmaceutically acceptable salts thereof), there are chemical entities of Formula I:

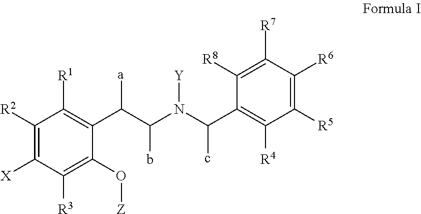

wherein:
R¹: (i) can be selected from the group consisting of H, C₁-C₄ alkyl, substituted C₁-C₄ alkyl, cyclopropyl, cyclobutyl, cyclopropylmethyl, 2-oxetanyl, 3-oxetanyl, OH, C₁-C₄ alkoxy, substituted C₁-C₄ alkoxy, C₁-C₄ alkylthio, substituted C₁-C₄ alkylthio, and halogen, if each of R² and R³ is independently selected from H, CH₃, or halogen; or (ii) together with R² can form an alkanediyl, alkenediyl, heteroalkanediyl, or heteroalkenediyl moiety, which moiety can further be substituted with a first substituent selected from the group consisting of C₁-C₄ alkyl, substituted C₁-C₄ alkyl, cyclopropyl, cyclobutyl, cyclopropylmethyl, 2-oxetanyl, 3-oxetanyl, C₁-C₄ alkoxy, C₁-C₄ alkylthio, OCF₃, OCHF₂, SCF₃, SCHF₂, CN, and halogen, and, if such moiety contains at least one additional H atom, a second substituent selected from the group consisting of C₁-C₄ alkyl, substituted C₁-C₄ alkyl, and halogen, if R³ is H, CH₃, or halogen. For example, if R³ is H, CH₃, or halogen, then R¹ and R² together can form CH₂CH₂, CH₂CH₂CH₂, CH₂CH₂CH₂CH₂, (CH₂)₅, CH═CHCH═CH, CH₂CH₂O, CH₂OCH₂, OCH₂CH₂, CH₂CH₂CH₂O, CH₂CH₂OCH₂, CH₂OCH₂CH₂, OCH₂CH₂CH₂, OCH═CH, CH═CHO, OCH₂O, OCH₂CH₂O, CH₂CH₂S, CH₂SCH₂, SCH₂CH₂, CH₂CH₂CH₂S, CH₂CH₂SCH₂, CH₂SCH₂CH₂, SCH₂CH₂CH₂, SCH═CH, CH═CHS, ON═CH, CH═NO, OCH═N, N═CHO, SN═CH, CH═NS, SCH═N, N═CHS, NHCH═CH, CH═CHNH, —NHN═CH, CH═NNH, NHCH═N, N═CHNH, CH═CHCH=N, CH═CHN═CH, CH═NCH═CH, or N═CHCH═CH; or (iii) together with b can form an alkanediyl, alkenediyl, or heteroalkanediyl moiety, which moiety can be substituted with a substituent selected from the group consisting of C₁-C₄ alkyl, substituted C₁-C₄ alkyl, cyclopropyl, cyclobutyl, cyclopropylmethyl, 2-oxetanyl, 3-oxetanyl, C₁-C₄ alkoxy, C₁-C₄ alkylthio, OCF₃, OCHF₂, SCF₃, SCHF₂, CN, and halogen, and, if such moiety contains at least one additional H atom, a second substituent selected from the group consisting of C₁-C₄ alkyl, substituted C₁-C₄ alkyl, and halogen, if each of R² and R³ is independently selected from H, CH₃, or halogen. For example, if R² and R³ are each independently H, CH₃, or halogen, then R¹ and b can form any one of the following non-limiting linkages: CH₂, CH₂CH₂, CH₂CH₂CH₂, CH═CH, CH₂CH═CH, CH═CHCH₂, OCH₂, OCH₂CH₂, CH₂OCH₂, SCH₂, SCH₂CH₂, and CH₂SCH₂;
R²: (i) is selected from the group consisting of H, C₁-C₄ alkyl, substituted C₁-C₄ alkyl, cyclopropyl, cyclobutyl, cyclopropylmethyl, 2-oxetanyl, 3-oxetanyl, OH, C₁-C₄ alkoxy, substituted C₁-C₄ alkoxy, C₁-C₄ alkylthio, substituted C₁-C₄ alkylthio, and halogen, if each of R¹ and R³ is independently selected from H, CH₃, or halogen; or (ii) together with R¹ can form an alkanediyl, alkenediyl, heteroalkanediyl, or heteroalkenediyl moiety, which moiety can be substituted with a first substituent selected from the group consisting of C₁-C₄ alkyl, substituted C₁-C₄ alkyl, cyclopropyl, cyclobutyl, cyclopropylmethyl, 2-oxetanyl, 3-oxetanyl, C₁-C₄ alkoxy, C₁-C₄ alkylthio, OCF₃, OCHF₂, SCF₃, SCHF₂, CN, and halogen, and, if such moiety contains at least one additional H atom, a second substituent selected from the group consisting of C₁-C₄ alkyl, substituted C₁-C₄ alkyl, and halogen, if R³ is H, CH₃, or halogen;
R³: (i) is selected from the group consisting of H, C₁-C₄ alkyl, substituted C₁-C₄ alkyl, cyclopropyl, cyclobutyl, cyclopropylmethyl, 2-oxetanyl, 3-oxetanyl, OH, C₁-C₄ alkoxy, substituted C₁-C₄ alkoxy, C₁-C₄ alkylthio, substituted C₁-C₄ alkylthio, and halogen, if each of R¹ and R² is independently selected from H, CH₃, or halogen; or (ii) together with Z can form an alkanediyl, alkenediyl, heteroalkanediyl, or heteroalkenediyl moiety, which moiety can be substituted with one selected from the group consisting of C₁-C₄ alkyl, substituted C₁-C₄ alkyl, cyclopropyl, cyclobutyl, cyclopropylmethyl, 2-oxetanyl, 3-oxetanyl, OH, C₁-C₄ alkoxy, C₁-C₄ alkylthio, OCF₃, OCHF₂, SCF₃, SCHF₂, CN, and halogen, if each of R¹ and R² is independently selected from the group consisting of H, CH₃, and halogen;

wherein:
one of R⁴, R⁵, R⁶, R⁷, and R³ is selected from the group consisting of R⁹, OR⁹, SR⁹, S(O)R⁹, S(O)₂R⁹, N(R⁹)C(O)R⁹, N(R⁹)C(O)OR⁹, N(R⁹)C(O)N(H)R⁹, N(R⁹)C(O)N(C₁-C₆ alkyl)R⁹, N(R⁹)S(O)₂R⁹, CH₂OR⁹, CH₂SR⁹, CH₂S(O)R⁹, CH₂S(O)₂R⁹, CH₂N(R⁹)C(O)R⁹, CH₂N(R⁹)C(O)OR⁹, CH₂N(R⁹)C(O)N(H)R⁹, CH₂N(R⁹)C(O)N(C₁-C₆ alkyl)R⁹, CH₂N(R⁹)S(O)₂R⁹, CH═NOR⁹, C(C₁-C₆ alkyl)═NOR⁹, C(O)OR⁹, C(O)N(H)R⁹, C(O)N(C₁-C₆ alkyl)R⁹, CH₂C(O)OR⁹, CH₂C(O)N(H)R⁹, CH₂C(O)N(C₁-C₆ alkyl)R⁹, CN, and halogen, a second one of R⁴, R⁵, R⁶, R⁷ or R³ is selected from the group consisting of H, C₁-C₄ alkyl, CHF₂, CF₃, OH, OCH₃, OC₂H₅, OCHF₂, OCF₃, and halogen, and the remainder of R⁴, R⁵, R⁶, R⁷, and R³ are each independently H, CH₃, or halogen;
or if R⁴ and R⁵ together form an alkanediyl, alkenediyl, heteroalkanediyl, or heteroalkenediyl moiety, which moiety can be substituted with a group selected from the group consisting of H, C₁-C₄ alkyl, substituted C₁-C₄ alkyl, cyclopropyl, cyclobutyl, cyclopropylmethyl, 2-oxetanyl, 3-oxetanyl, C₁-C₄ alkoxy, OCF₃, C₁-C₄ alkylthio, CN, and halogen, then each of R⁶, R⁷, and R³ is independently selected from the group consisting of H, CH₃, and halogen. Non-limiting examples of alkanediyl moieties include CH₂CH₂, CH₂CH₂CH₂, and CH₂CH₂CH₂CH₂. A non-limiting example of an alkenediyl moiety is CH═CHCH═CH. Non-limiting examples of heteroalkanediyl moieties include CH₂CH₂O, CH₂OCH₂, OCH₂CH₂, CH₂CH₂CH₂O, CH₂CH₂OCH₂, CH₂OCH₂CH₂, OCH₂CH₂CH₂, OCH₂O, OCH₂CH₂O, CH₂CH₂S, CH₂SCH₂, SCH₂CH₂, CH₂CH₂CH₂S, CH₂CH₂SCH₂, CH₂SCH₂CH₂, SCH₂CH₂CH₂, NHCH₂CH₂, and NHCH₂NH. Non-limiting examples of heteroalkenediyl moieties include OCH═CH, CH═CHO, SCH═CH, CH═CHS, ON═CH, CH═NO, OCH═N, N═CHO, SN═CH, CH═NS, SCH═N, N═CHS, NHCH═CH, CH═CHNH, NHN═CH, CH═NNH, NHCH═N, N═CHNH, CH═CHCH=N, CH═CHN═CH, CH═NCH═CH, and N═CHCH═CH;
or if R⁴ and b together form an alkanediyl, alkenediyl, or heteroalkanediyl moiety, which moiety can be substituted with (a) a first group selected from the group consisting of C₁-C₄ alkyl, substituted C₁-C₄ alkyl, cyclopropyl, cyclobutyl, cyclopropylmethyl, 2-oxetanyl, 3-oxetanyl, C₁-C₄ alkoxy, C₁-C₄ alkylthio, OCF₃, OCHF₂, SCF₃, SCHF₂, CN, and halogen, and (b) a second group selected from the group consisting of C₁-C₄ alkyl, substituted C₁-C₄ alkyl, and halogen, then one of R⁵, R⁶, R⁷, and R³ is selected from the group consisting of H, C₁-C₄ alkyl, CHF₂, CF₃, OH, OCH₃, OC₂H₅, OCHF₂, OCF₃, and halogen, and the remainder of R⁵, R⁶, R⁷, and R³ are each independently selected from the group consisting of H, CH₃, and halogen. Non-limiting examples of alkanediyl moieties include CH₂ and CH₂CH₂. A non-limiting example of an alkenediyl moiety is CH═CH. Non-limiting examples of heteroalkanediyl moieties include OCH₂ and SCH₂;
or if R⁴ and c together form an alkanediyl, alkenediyl, or heteroalkanediyl moiety, then one of R⁵, R⁶, R⁷, and R³ is selected from the group consisting of H, C₁-C₄ alkyl, CHF₂, CF₃, OH, OCH₃, OC₂H₅, OCHF₂, OCF₃, CN, and halogen, and the remainder of R⁵, R⁶, R⁷, and R³ are each independently selected from the group consisting of H, CH₃, and halogen. Non-limiting examples of alkanediyl moieties include CH₂, CH₂CH₂, and CH₂CH₂CH₂. Non-limiting examples of alkenediyl moieties include CH═CH, CH₂CH═CH, and CH═CHCH₂. Non-limiting examples of heteroalkanediyl moieties include CH₂OCH₂, OCH₂CH₂, CH₂SCH₂, and SCH₂CH₂;

wherein:
X is selected from the group consisting of CN, C(O)NH₂, C(O)N(H)R⁹, C(O)N(C₁-C₆ alkyl)R⁹, C(O)(C₄-C₆ heterocyclyl), CHF₂, CF₃, OH, O(C₁-C₆ alkyl), OCHF₂, OCF₃, S(C₁-C₆ alkyl), SCF₃, SCHF₂, F, Cl, aryl, and heteroaryl generally, but otherwise selected from the group consisting of CN, C(O)NH₂, C(O)N(H)R⁹, C(O)N(C₁-C₆ alkyl)R⁹, C(O)(C₄-C₆ heterocyclyl), CHF₂, CF₃, OH, O(C₁-C₆ alkyl), OCHF₂, OCF₃, S(C₁-C₆ alkyl), SCF₃, SCHF₂, aryl, and heteroaryl in other instances if Z is alkyl and R⁴ is hydroxy;
Y is selected from the group consisting of H, CH₃, C(O)R⁹, CH₂OC(O)R⁹, and C(O)OR⁹;
Z: (i) is selected from the group consisting of H, C₁-C₁₀ alkyl, substituted C₁-C₁₀ alkyl, C₁-C₁₀ heteroalkyl, C₂-C₁₀ alkenyl, C₂-C₁₀ heteroalkenyl, C₂-C₁₀ alkynyl, C₃-C₆ cycloalkyl, (C₃-C₆ cycloalkyl)(C₁-C₃ alkyl), (C₃-C₆ cycloalkyl)(C₁-C₃ heteroalkyl), C₄-C₆ heterocyclyl, (C₄-C₆ heterocyclyl)(C₁-C₃ alkyl), (C₄-C₆ heterocyclyl)(C₁-C₈ heteroalkyl), aryl(C₁—C alkyl), aryl(C₁—C heteroalkyl), heteroaryl(C₁-C₃ alkyl), heteroaryl(C₁-C₃ heteroalkyl), C(O)R⁹, C(O)OR⁹, CH₂OC(O)R⁹, C(O)NH₂, C(O)N(H)R⁹, C(O)N(C₁-C₆ alkyl)R⁹, and C(O)(C₄-C₆ heterocyclyl); or (ii) together with R³ form an alkanediyl, alkenediyl, heteroalkanediyl, or heteroalkenediyl moiety, which moiety can further be substituted with one group selected from the group consisting of C₁-C₄ alkyl, substituted C₁-C₄ alkyl, cyclopropyl, cyclobutyl, cyclopropylmethyl, 2-oxetanyl, 3-oxetanyl, OH, C₁-C₄ alkoxy, C₁-C₄ alkylthio, OCF₃, CN, and halogen. Examples of alkanediyl moieties include CH₂CH₂ and CH₂CH₂CH₂; non-limiting examples of alkenediyl moieties include CH═CH; non-limiting examples of heteroalkanediyl moieties include CH₂O, CH₂CH₂O, CH₂S, and CH₂CH₂S; and non-limiting examples of heteroalkenediyl moieties include CH═N;

wherein:
with regard to a, b, and c: (i) two of a, b, and c are each independently selected from the group consisting of H, CH₃, and C₂H₅, while the third of a, b, and c is H; or (ii) b and c together are CH₂, CH₂CH₂, CH₂CH₂CH₂, or CH₂OCH₂, while a is H or CH₃; or (iii) if b and R¹ together form an alkanediyl, alkenediyl, or heteroalkanediyl moiety, which moiety can be substituted with a first substituent selected from the group consisting of C₁-C₄ alkyl, substituted C₁-C₄ alkyl, cyclopropyl, cyclobutyl, cyclopropylmethyl, 2-oxetanyl, 3-oxetanyl, C₁-C₄ alkoxy, C₁-C₄ alkylthio, OCF₃, OCHF₂, SCF₃, SCHF₂, CN, and halogen, and, if the moiety contains at least one additional H atom, a second substituent selected from the group consisting of C₁-C₄ alkyl, substituted C₁-C₄ alkyl, and halogen, then one of a and c is H, CH₃, or C₂H₅, and the other of a and c is H. Non-limiting examples of alkanediyl moieties include CH₂, CH₂CH₂, and CH₂CH₂CH₂. Non-limiting examples of alkenediyl moieties include CH═CH, CH₂CH═CH, and CH═CHCH₂. Non-limiting examples of heteroalkanediyl moieties include CH₂O, CH₂CH₂O, CH₂OCH₂, CH₂S, CH₂CH₂S, and CH₂SCH₂; or (iv) if b and R⁴ together form an alkanediyl, alkenediyl, or heteroalkanediyl moiety, which moiety can be substituted with a first substituent selected from the group consisting of H, C₁-C₄ alkyl, substituted C₁-C₄ alkyl, cyclopropyl, cyclobutyl, cyclopropylmethyl, 2-oxetanyl, 3-oxetanyl, C₁-C₄ alkoxy, C₁-C₄ alkylthio, OCF₃, OCHF₂, SCF₃, SCHF₂, CN, and halogen, and, if the moiety contains at least one additional H atom, a second substituent selected from the group consisting of C₁-C₄ alkyl, substituted C₁-C₄ alkyl, and halogen, then one of a and c is H, CH₃, and C₂H₅, and the other of a and c is H. Non-limiting examples of alkanediyl moieties include CH₂ and CH₂CH₂. A non-limiting example of an alkenediyl moiety is CH═CH. Non-limiting examples of heteroalkanediyl moieties include CH₂O and CH₂S; or (v) if c and R⁴ together form an alkanediyl, alkenediyl, or heteroalkanediyl moiety, then a and b are each independently selected from H or CH₃. Non-limiting examples of alkanediyl moieties include CH₂, CH₂CH₂, and CH₂CH₂CH₂. Non-limiting examples of alkenediyl moieties include CH═CH, and CH₂CH═CH. Non-limiting examples of heteroalkyl moieties include CH₂O, CH₂OCH₂, CH₂CH₂O, CH₂S, CH₂SCH₂, and CH₂CH₂S;

and wherein:
R⁹ is, independently for each occurrence, selected from the group consisting of H, C₁-C₆ alkyl, C₁-C₆ heteroalkyl, substituted C₁-C₆ alkyl, substituted C₁-C₆ heteroalkyl, C₂-C₆ alkenyl, C₂-C₆ heteroalkenyl, C₂-C₆ alkynyl, C₃-C₆ cycloalkyl, (C₃-C₆ cycloalkyl)(C₁-C₆ alkyl), (C₃-C₆ cycloalkyl)(C₁-C₆ heteroalkyl), C₃-C₆ heterocyclyl, (C₃-C₆ heterocyclyl)(C₁-C₆ alkyl), (C₃-C₆ heterocyclyl)(C₁-C₆ heteroalkyl), aryl, aryl(C₁-C₆ alkyl), aryl(C₁-C₆ heteroalkyl), heteroaryl, heteroaryl(C₁-C₆ alkyl), and heteroaryl(C₁-C₆ heteroalkyl).

### Examples of Embodiments of Chemical Entities

Examples of chemical entities of Formula I are provided in Table 1 below.

TABLE 1
Examples of Compounds of Formula I *

| # | X | Y | Z | R³ | R² | R¹ | a | b | c | R⁴ | R⁵ | R⁶ | R⁷ | R⁸ |
|---|---|---|---|---|---|---|---|---|---|---|---|---|---|---|
| 1 | CN | H | Me | H | H | H | H | H | H | H | H | H | H | H |
| 2 | CN | H | Me | H | H | F | H | H | H | H | H | H | H | H |
| 3 | CN | H | Me | H | F | H | H | H | H | H | H | H | H | H |
| 4 | CN | H | Me | H | F | H | H | H | H | OH | H | H | H | H |
| 5 | CN | H | Me | F | H | F | H | H | H | H | H | H | H | H |
| 6 | CN | H | Me | H | H | H | H | H | H | F | H | H | H | H |
| 7 | CN | H | Me | H | H | H | H | H | H | H | F | H | H | H |
| 8 | CN | H | Me | H | H | H | H | H | H | H | H | F | H | H |
| 9 | CN | H | Me | H | H | Cl | H | H | H | H | H | H | H | H |
| 10 | CN | H | Me | H | Cl | H | H | H | H | H | H | H | H | H |
| 11 | CN | H | Me | Cl | H | H | H | H | H | H | H | H | H | H |
| 12 | CN | H | Me | H | H | H | H | H | H | Cl | H | H | H | H |
| 13 | CN | H | Me | H | H | H | H | H | H | H | Cl | H | H | H |
| 14 | CN | H | Me | H | H | H | H | H | H | H | H | Cl | H | H |
| 15 | CN | H | Me | H | H | Me | H | H | H | H | H | H | H | H |
| 16 | CN | H | Me | H | Me | H | H | H | H | H | H | H | H | H |
| 17 | CN | H | Me | Me | H | H | H | H | H | H | H | H | H | H |
| 18 | CN | H | Me | H | H | H | H | H | H | Me | H | H | H | H |
| 19 | CN | H | Me | H | H | H | H | H | H | H | Me | H | H | H |
| 20 | CN | H | Me | H | H | H | H | H | H | H | H | Me | H | H |
| 21 | CN | H | Me | H | H | CF₃ | H | H | H | H | H | H | H | H |
| 22 | CN | H | Me | H | CF₃ | H | H | H | H | H | H | H | H | H |
| 23 | CN | H | Me | CF₃ | H | H | H | H | H | H | H | H | H | H |
| 24 | CN | H | Me | H | H | OMe | H | H | H | H | H | H | H | H |
| 25 | CN | H | Me | H | OMe | H | H | H | H | H | H | H | H | H |
| 26 | CN | H | Me | OMe | H | H | H | H | H | H | H | H | H | H |
| 27 | CN | H | Me | H | H | H | H | H | H | OMe | H | H | H | H |
| 28 | CN | H | Me | H | H | H | H | H | H | H | OMe | H | H | H |
| 29 | CN | H | Me | H | H | H | H | H | H | H | H | OMe | H | H |
| 30 | CN | H | Me | H | (CH═CH)₂ | | H | H | H | Cl | H | H | H | H |
| 31 | CN | H | Me | H | CH₂CH₂O | | H | H | H | Cl | H | H | H | H |
| 32 | CN | H | Me | H | CH═CHO | | H | H | H | Cl | H | H | H | H |
| 33 | CN | H | Me | H | OCH₂O | | H | H | H | Cl | H | H | H | H |
| 34 | CN | H | Me | H | CH═CHNH | | H | H | H | Cl | H | H | H | H |
| 35 | CN | H | Me | H | CH═NO | | H | H | H | Cl | H | H | H | H |
| 36 | CN | H | Me | CHF₂ | H | H | H | H | H | H | H | H | H | H |
| 37 | CN | H | H | H | H | H | H | H | H | Cl | H | H | H | H |
| 38 | CN | H | CHF₂ | H | H | H | H | H | H | Cl | H | H | H | H |
| 39 | CN | H | CF₃ | H | H | H | H | H | H | Cl | H | H | H | H |
| 40 | CN | H | Et | H | H | H | H | H | H | Cl | H | H | H | H |
| 41 | CN | H | Bu | H | H | H | H | H | H | Cl | H | H | H | H |
| 42 | CN | H | cpm | H | H | H | H | H | H | Cl | H | H | H | H |
| 43 | CN | H | CH₂CH₂ | | H | H | H | H | H | Cl | H | H | H | H |
| 44 | CN | H | CH═CH | | H | H | H | H | H | Cl | H | H | H | H |
| 45 | CN | H | CH₂O | | H | H | H | H | H | Cl | H | H | H | H |
| 46 | CN | H | CH₂CH₂O | | H | H | H | H | H | Cl | H | H | H | H |
| 47 | CN | H | CH═N | | H | H | H | H | H | Cl | H | H | H | H |
| 48 | CN | H | N═CH | | H | H | H | H | H | Cl | H | H | H | H |
| 49 | CN | H | Me | H | H | H | H | H | CH₂ | | H | H | H | H |
| 50 | CN | H | Me | H | H | H | H | H | CH₂CH₂ | | H | H | H | H |
| 51 | CN | H | Me | H | H | H | H | H | (CH₂)₃ | | H | H | H | H |
| 52 | CN | H | Me | H | H | H | H | H | CH₂O | | H | H | H | H |
| 53 | CN | H | Me | H | H | H | H | H | CH₂OCH₂ | | H | H | H | H |
| 54 | CN | H | Me | H | H | H | H | H | CH₂CH₂O | | H | H | H | H |
| 55 | CN | H | Me | H | H | H | H | H | H | OH | H | H | H | H |
| 56 | CN | H | Me | H | H | H | H | H | H | H | OH | H | H | H |
| 57 | CN | H | Me | H | H | H | H | H | H | H | H | OH | H | H |
| 58 | CN | H | Me | H | H | H | H | H | H | Cl | H | H | H | Cl |
| 59 | CN | H | Me | H | H | H | H | H | H | F | H | F | H | H |
| 60 | CN | H | Me | H | H | H | H | H | H | Cl | H | H | H | Cl |
| 61 | CN | H | Me | H | H | H | H | H | H | d | H | H | H | H |
| 62 | CN | H | Me | H | H | H | H | H | H | e | H | H | H | H |
| 63 | CN | H | Me | H | H | H | H | H | H | CH₂CH₂O | | H | H | H |
| 64 | CN | H | Me | H | H | H | H | H | H | CH═CHO | | H | H | H |
| 65 | CN | H | Me | H | H | H | H | H | H | OCH₂O | | H | H | H |
| 66 | CN | H | Me | H | H | H | H | H | H | CH═CHNH | | H | H | H |
| 67 | CN | H | Me | H | H | H | H | H | H | CH═NO | | H | H | H |
| 68 | CN | H | Me | H | H | H | H | H | H | OCH₂CH₂ | | H | H | H |
| 69 | CN | H | Me | H | H | H | H | H | H | OCH═CH | | H | H | H |
| 70 | CN | H | H | H | H | H | H | H | H | SCH═CH | | H | H | H |
| 71 | CN | H | H | H | H | H | H | H | H | NHCH═CH | | H | H | H |
| 72 | CN | H | Me | H | H | H | H | H | H | NHC(F)═N | | H | H | H |
| 73 | CN | H | Me | H | H | H | H | H | H | N(Me)CH═N | | H | H | H |
| 74 | CN | H | Me | H | H | H | H | H | H | ON═CH | | H | H | H |
| 75 | CN | H | Me | H | H | H | H | H | H | N═CH—CH═CH | | H | H | H |
| 76 | CN | H | Me | H | H | H | H | H | H | N═CH—CH═CH | | H | F | H |
| 77 | CN | H | Me | H | H | H | H | H | H | Cl | H | OH | H | H |
| 78 | CN | H | Me | H | H | H | H | H | H | CN | H | H | H | H |
| 79 | CN | H | Me | H | H | H | H | H | H | H | H | H | H | H |
| 80 | CN | H | Me | H | H | H | Me | H | H | Cl | H | H | H | H |
| 81 | CN | H | Me | H | H | H | H | Me | H | Cl | H | H | H | H |
| 82 | CN | H | Me | H | H | H | H | H | Me | Cl | H | H | H | H |
| 83 | CN | H | Me | H | H | H | H | CH₂ | | Cl | H | H | H | H |
| 84 | CN | H | Me | H | H | H | H | CH₂CH₂ | | Cl | H | H | H | H |
| 85 | CN | H | Me | H | H | H | H | CH₂CH₂ | | OH | H | H | H | H |
| 86 | CN | H | Me | H | H | H | H | (CH₂)₃ | | Cl | H | H | H | H |
| 87 | CN | H | Me | H | H | H | H | CH₂OCH₂ | | Cl | H | H | H | H |
| 88 | CN | Me | Me | H | H | H | H | H | H | Cl | H | H | H | H |
| 89 | CN | Ac | Me | H | H | H | H | H | H | Cl | H | H | H | H |
| 90 | CN | Bz | Me | H | H | H | H | H | H | Cl | H | H | H | H |
| 91 | CN | f | Me | H | H | H | H | H | H | Cl | H | H | H | H |
| 92 | d | H | Me | H | H | H | H | H | H | Cl | H | H | H | H |
| 93 | CF₃ | H | Me | H | H | H | H | H | H | OH | H | H | H | H |
| 94 | CHF₂ | H | Me | H | H | H | H | H | H | Cl | H | H | H | H |
| 95 | OH | H | Me | H | H | H | H | H | H | Cl | H | H | H | H |
| 96 | OMe | H | Me | H | H | H | H | H | H | OH | H | H | H | H |
| 97 | OCHF₂ | H | Me | H | H | H | H | H | H | Cl | H | H | H | H |
| 98 | OCF₃ | H | Me | H | H | H | H | H | H | Cl | H | H | H | H |
| 99 | SCF₃ | H | Me | H | H | H | H | H | H | Cl | H | H | H | H |
| 100 | g | H | Me | H | H | H | H | H | H | Cl | H | H | H | H |
| 101 | 4-isoxazolyl | H | Me | H | H | H | H | H | H | Cl | H | H | H | H |
| 102 | 2-oxazolyl | H | Me | H | H | H | H | H | H | Cl | H | H | H | H |
| 103 | 2-thiazolyl | H | Me | H | H | H | H | H | H | Cl | H | H | H | H |
| 104 | 2-pyrimidyl | H | Me | H | H | H | H | H | H | Cl | H | H | H | H |
| 105 | 5-pyrimidyl | H | Me | H | H | H | H | H | H | Cl | H | H | H | H |
| 106 | 3-pyridyl | H | Me | H | H | H | H | H | H | Cl | H | H | H | H |
| 107 | 4-pyridyl | H | Me | H | H | H | H | H | H | Cl | H | H | H | H |
| 108 | CN | H | Me | H | H | H | H | H | H | SMe | H | H | H | H |
| 109 | CN | H | H | H | H | H | H | H | H | SMe | H | H | H | H |

| | | | | | | | | | | | | | | |
|---|---|---|---|---|---|---|---|---|---|---|---|---|---|---|
| # | X | Y | Z | R³ | R² | R¹ | b | a | c | R⁴ | R⁵ | R⁶ | R⁷ | R⁸ |

| | | | | | | | | | | | | | |
|---|---|---|---|---|---|---|---|---|---|---|---|---|---|
| 110 | CN | H | Me | H | H | CH₂ | H | H | OH | H | H | H | H |

| | | | | | | | | | | | | | | |
|---|---|---|---|---|---|---|---|---|---|---|---|---|---|---|
| # | X | Y | Z | R³ | R² | R¹ | a | c | b | R⁴ | R⁵ | R⁶ | R⁷ | R⁸ |

| | | | | | | | | | | | | | |
|---|---|---|---|---|---|---|---|---|---|---|---|---|---|
| 111 | CN | H | Me | H | H | H | H | H | CH₂O | H | H | H | H |

| # | X | Y | Z | R³ | R² | R¹ | a | b | c | R⁴ | R⁵ | R⁶ | R⁷ | R⁸ |
|---|---|---|---|---|---|---|---|---|---|---|---|---|---|---|
| 112 | OMe | H | Me | H | H | H | H | H | H | H | H | H | H | OH |
| 113 | CN | H | Me | H | H | H | H | H | H | H | H | H | H | OH |
| 114 | CN | H | H | H | H | H | H | H | H | H | H | H | H | OH |
| 115 | CN | H | CF₃ | H | H | H | H | H | H | H | H | H | H | OH |
| 116 | CF₃ | H | H | H | H | H | H | H | H | OH | H | H | H | H |
| 117 | CN | H | Me | H | F | H | H | H | H | H | H | H | H | OH |
| 118 | CN | Me | Me | H | H | H | H | H | H | H | H | H | H | Cl |
| 119 | OMe | H | H | H | H | H | H | H | H | H | H | H | H | SMe |
| 120 | OH | H | H | H | H | H | H | H | H | H | H | H | H | SMe |
| 121 | SMe | H | H | H | H | H | H | H | H | H | H | H | H | SMe |
| 122 | SMe | H | H | H | H | H | H | H | H | H | H | H | H | OH |
| 123 | CHF₂ | H | H | H | H | H | H | H | H | H | H | H | H | SMe |
| 124 | CN | H | H | H | H | H | H | H | H | OCF₃ | H | H | H | H |
| 125 | hexyl | H | Me | H | H | H | H | H | H | H | H | H | H | OH |
| 126 | Benzyl | H | Me | H | H | H | H | H | H | H | H | H | H | OH |
| 127 | O-Phenyl | H | Me | H | H | H | H | H | H | H | H | H | H | OH |
| 128 | CN | H | Me | H | F | H | H | H | H | H | H | H | H | SMe |
| 129 | CN | H | Me | H | F | H | H | H | H | H | H | H | H | OH |
| 130 | F | H | H | H | H | H | H | H | H | H | H | H | H | OMe |
| 131 | Cl | H | H | H | H | H | H | H | H | H | H | H | H | OH |

| | | | | | | | | | | | | | |
|---|---|---|---|---|---|---|---|---|---|---|---|---|---|
| 132 | H | H | H | H | F | H | H | H | H | OCH₂O | H | H | H |

Abbreviations used in the table above have the following meanings: Ac=acetyl; Bz=benzoyl; Eu=butyl; cpm=cyclopropylmethyl; d=C(O)N(CH₃)₂; e=S(O)₂NH₂; Et=ethyl; f=C(O)OMe; g=C(O)NHCH₃; Me=methyl.

### Pharmacology

### Serotonin Receptor 5-HT₂ Functional Assays

Non-limiting examples of methods of measuring serotonin receptor functional activation are described as follows.

To measure serotonin receptor functional activation, either Gq dissociation by bioluminescence resonance energy transfer (BRET) or Gq-dependent calcium flux was performed for selected compounds. To measure 5-HT₂ receptor-mediated Gq activation via Gq/γ1 dissociation as measured by BRET (McCorvy J D, Wacker D, Wang S, Agegnehu B, Liu J, Lansu K, Tribo A R, Olsen R H J, Che T, Jin J, Roth B L. Structural determinants of 5-HT_{2B} receptor activation and biased agonism. Nat Struct Mol Biol. 2018; 25(9): 787-96), HEK293T cells were sub-cultured in Dulbecco's Modified Eagle Medium (DMEM) supplemented with 10% dialyzed fetal bovine serum (FBS) and were co-transfected in a 1:1:1:1 ratio with RLuc8-fused human Gaq (Gaq-RLuc8), a GFP²-fused to the C-terminus of human Gy1(Gγ1-GFP²), human Gβ1, and 5-HT₂ receptor using TransiT-2020. After at least 18-24 hours, transfected cells were plated in poly-lysine coated 96-well white clear bottom cell culture plates in DMEM containing 1% dialyzed FBS at a density of 25,000-40,000 cells in 200 μL per well and incubated overnight. The following day, medium was decanted, and cells were washed with 60 μL of drug buffer (1× HBSS, 20 mM HEPES, pH 7.4), then 60 μL of drug buffer was added per well. Cells were pre-incubated at in a humidified atmosphere at 37° C. before receiving drug stimulation. Drug stimulation utilized 30 μL addition of drug (3×) diluted in McCorvy buffer (1× HBSS, 20 mM HEPES, pH 7.4, supplemented with 0.3% BSA fatty acid free, 0.03% ascorbic acid) and plates were incubated at for 1 hour at 37° C. Substrate addition occurred 15 minutes before reading and utilized 10 μL of the RLuc substrate coelenterazine 400a for Gq dissociation BRET² (Prolume/Nanolight, 5 μM final concentration). Plates were read for luminescence at 400 nm and fluorescent GFP² emission at 510 nm at 1 second per well using a Mithras L B940 multimode microplate reader (e.g. one provided by Berthold). The BRET ratios of fluorescence/luminescence were calculated per well and were plotted as a function of drug concentration using Graphpad Prism 8 (Graphpad Software Inc., San Diego, CA). Data were normalized to % 5-HT stimulation and analyzed using nonlinear regression “log(agonist) vs. response” to yield Eₘₐₓ and EC₅₀ parameter estimates.

Calcium flux was measured using stable-expressing 5-HT₂ Flp-In 293 T-Rex Tetracycline inducible system by methods known in the art (e.g. Investigation of the Structure-Activity Relationships of Psilocybin Analogues, ACS Pharmacol. Transl. Sci. 2020, Publication Date: Dec. 14, 2020, https://doi.org/10.1021/acsptsci.0c00176). Cell lines were maintained in DMEM containing 10% FBS, 10 μg/mL Blasticidin, and 100 μg/mL hygromycin B. At least 20-24 hours before the assay, receptor expression was induced with tetracycline (2 μg/mL), and cells were seeded into 384-well poly-L-lysine-coated black plates at a density of 7,500 cells/well in DMEM containing 1% dialyzed FBS. On the day of the assay, the cells were incubated for 1 hour at 37° C. with Fluo-4 Direct dye (Invitrogen, 20 μL/well) reconstituted in drug buffer (20 mM HEPES-buffered HBSS, pH 7.4) containing 2.5 mM probenecid. Drug dilutions were prepared at 5× final concentration in McCorvy buffer (20 mM HEPES-buffered HBSS, 0.1% BSA, 0.01% ascorbic acid, pH 7.4). After dye load, cells were allowed to equilibrate to room temperature for 15 minutes, and then placed in a FLIPR^{TETRA} fluorescence imaging plate reader (Molecular Devices). The FLIPR^{TETRA} was programmed to read baseline fluorescence for 10 s (1 read/s), and afterward 5 μL of drug per well was added, and fluorescence was read for a total of 5-10 min (1 read/s). Fluorescence in each well was normalized to the average of the first 10 reads for baseline fluorescence, and then either maximum-fold peak increase over baseline or area under the curve (AUC) was calculated. Either peak or AUC was plotted as a function of drug concentration, and data were normalized to percent 5-HT stimulation. Data were plotted, and non-linear regression was performed using “log(agonist) vs. response” in Graphpad Prism 8 to yield Eₘax and EC₅₀ parameter estimates.

The functional activity of various compounds disclosed herein at each of the 5-HT_{2A}, 5-HT_{2B}, and 5-HT_{2C} receptors was measured against and relative to the functional activity of 4-hydroxytryptamine at those receptors. A comparison of the functional activities is provided in Table 2 as follows. At least three replicate trials were performed for each compound:

TABLE 2

| | | 5-HT_{2A} BRET | | 5-HT_{2B} BRET | | 5-HT_{2C} BRET | |
|---|---|---|---|---|---|---|---|
| Compound | Identification | EC₅₀ | | EC₅₀ | | EC₅₀ | |
| — | — | (nM) | Eff % | (nM) | Eff % | (nM) | Eff % |
| 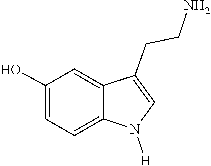 [SMILES: NCCc1c[nH]c2ccc(O)cc12] | 5- hydroxy- tryptamine (serotonin) (reference compound) | 2.9 | 100 | 0.8 | 100 | 0.35 | 99 |
| 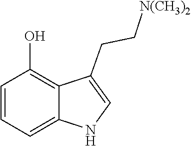 [SMILES: CN(C)CCc1c[nH]c2cccc(O)c12] | psilocin (reference compound) | 8.34 | 82 | 1.07 | 63 | 7.79 | 95 |
| 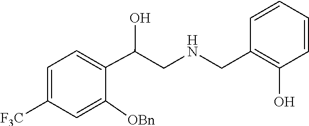 [SMILES: Oc1ccccc1CNCC(O)c1ccc(C(F)(F)F)cc1OCc1ccccc1] | Reference Compound 1a (reference compound) | 451 | 91 | 342 | 56 | 326 | 105 |
| 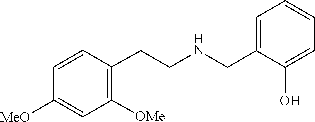 [SMILES: COc1ccc(CCNCc2ccccc2O)c(OC)c1] | Cpd #96 (reference compound) | 2.3 | 95 | Inactive | | 7.2 | 109 |
| 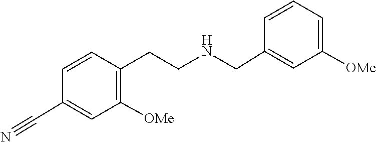 [SMILES: COc1cccc(CNCCc2ccc(C#N)cc2OC)c1] | Cpd #28 | 84 | 68 | Inactive | | 873 | 91 |
| 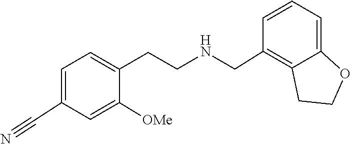 [SMILES: COc1cc(C#N)ccc1CCNCc1cccc2c1CCO2] | Cpd #63 | 135 | 62 | Inactive | | 2090 | 78 |
| 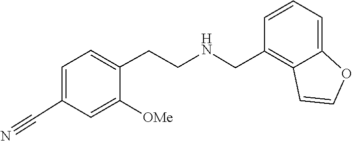 [SMILES: COc1cc(C#N)ccc1CCNCc1cccc2occc12] | Cpd #64 | 29.8 | 82 | 8920 | 93 | 357 | 104 |
| 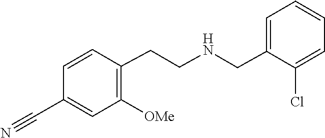 [SMILES: COc1cc(C#N)ccc1CCNCc1ccccc1Cl] | Cpd #12 | 102 | 90 | Inactive | | 1200 | 130 |
| 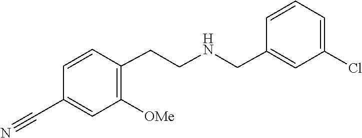 [SMILES: COc1cc(C#N)ccc1CCNCc1cccc(Cl)c1] | Cpd #13 | 393 | 69 | Inactive | | 1840 | 96 |
| 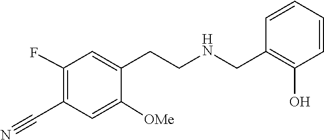 [SMILES: COc1cc(C#N)c(F)cc1CCNCc1ccccc1O] | Cpd #4 | 2.4 | 99 | 4090 | 73 | 37.5 | 115 |
| 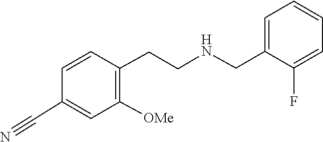 [SMILES: COc1cc(C#N)ccc1CCNCc1ccccc1F] | Cpd #6 | 381 | 81 | Inactive | | 2120 | 130 |
| 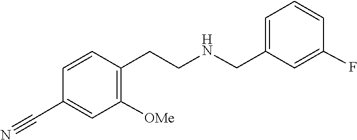 [SMILES: COc1cc(C#N)ccc1CCNCc1cccc(F)c1] | Cpd #7 | 133 | 84 | Inactive | | 800 | 126 |
| 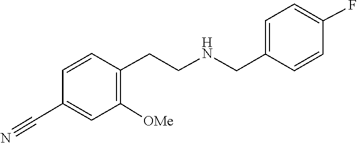 [SMILES: COc1cc(C#N)ccc1CCNCc1ccc(F)cc1] | Cpd #8 | 13100 | 52 | Inactive | | 8740 | 79 |
| 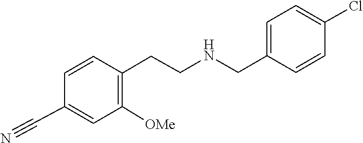 [SMILES: COc1cc(C#N)ccc1CCNCc1ccc(Cl)cc1] | Cpd #14 | 14300 | 40 | Inactive | | 13300 | 74 |
| 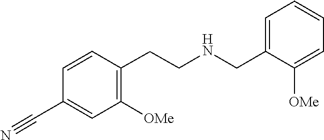 [SMILES: COc1cc(C#N)ccc1CCNCc1ccccc1OC] | Cpd #27 | 4.2 | 96 | 423 | 66 | 15.9 | 109 |
| 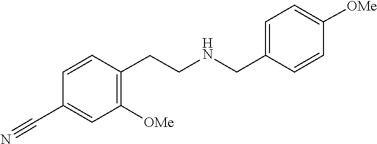 [SMILES: COc1ccc(CNCCc2ccc(C#N)cc2OC)cc1] | Cpd #29 | 3950 | 59 | Inactive | | 924 | 75 |
| 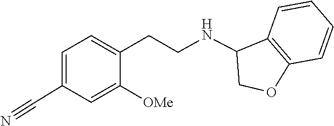 [SMILES: COc1cc(C#N)ccc1CCNC1COc2ccccc21] | Cpd #52 | 1040 | 69 | Inactive | | 12100 | 125 |
| 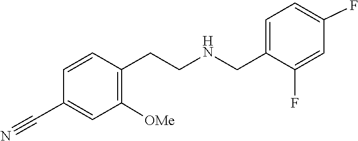 [SMILES: COc1cc(C#N)ccc1CCNCc1ccc(F)cc1F] | Cpd #59 | 14100 | 58 | Inactive | | 9930 | 94 |
| 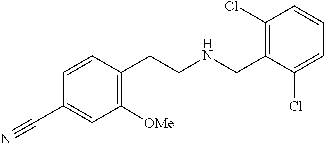 [SMILES: COc1cc(C#N)ccc1CCNCc1c(Cl)cccc1Cl] | Cpd #58 | 496 | 79 | Inactive | | 2170 | 85 |
| 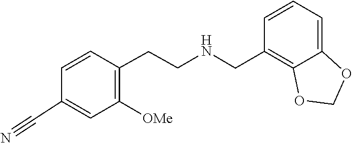 [SMILES: COc1cc(C#N)ccc1CCNCc1cccc2c1OCO2] | Cpd #65 | 19.7 | 84 | Inactive | | 496 | 97 |
| 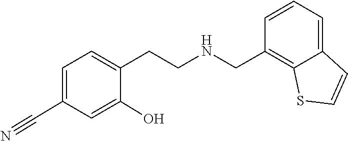 [SMILES: N#Cc1ccc(CCNCc2cccc3ccsc23)c(O)c1] | Cpd #70 | 0.3 | 11 | Inactive | | 5400 | 160 |
| 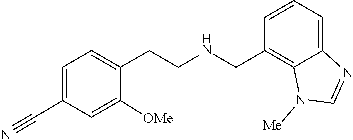 [SMILES: COc1cc(C#N)ccc1CCNCc1cccc2ncn(C)c12] | Cpd #73 | 73 | 4 | Inactive | | 0.04 | 7 |
| 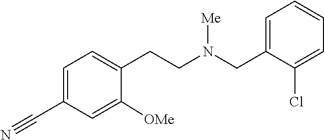 [SMILES: COc1cc(C#N)ccc1CCN(C)Cc1ccccc1Cl] | Cpd #88 | 0.01 | 14 | 4980 | 20 | 310000 | 86 |
| 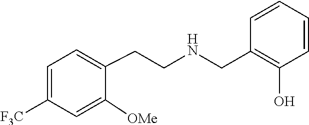 [SMILES: COc1cc(C(F)(F)F)ccc1CCNCc1ccccc1O] | Cpd #93 | 0.7 | 96 | 62.3 | 53 | 1.1 | 105 |
| 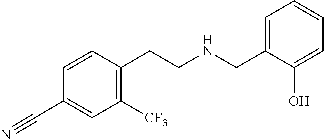 [SMILES: N#Cc1ccc(CCNCc2ccccc2O)c(C(F)(F)F)c1] | Cpd #112 | 432 | 90 | Inactive | | 2.6 | 83 |
| 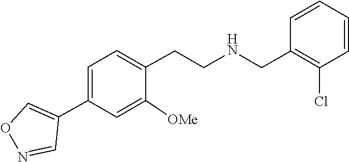 [SMILES: COc1cc(-c2cnoc2)ccc1CCNCc1ccccc1Cl] | Cpd #101 | 270 | 65 | Inactive | | 9520 | 116 |
| 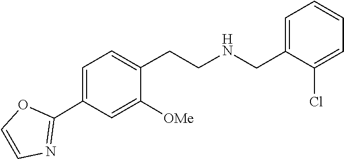 [SMILES: COc1cc(-c2ncco2)ccc1CCNCc1ccccc1Cl] | Cpd #102 | 2090 | 61 | 5370 | 143 | 1640 | 101 |
| 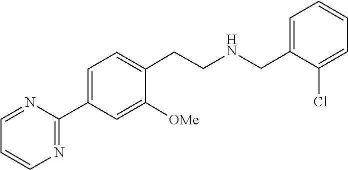 [SMILES: COc1cc(-c2ncccn2)ccc1CCNCc1ccccc1Cl] | Cpd #104 | 4890 | 71 | Inactive | | 5690 | 106 |
| 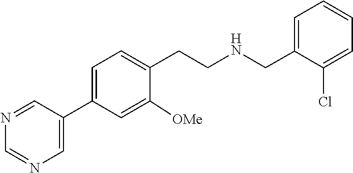 [SMILES: COc1cc(-c2cncnc2)ccc1CCNCc1ccccc1Cl] | Cpd #105 | 1610 | 86 | Inactive | | 1490 | 107 |
| 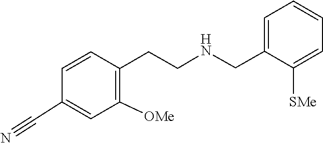 [SMILES: COc1cc(C#N)ccc1CCNCc1ccccc1SC] | Cpd #108 | 2.4 | 99 | 2380 | 128 | 16 | 103 |
| 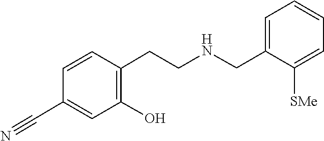 [SMILES: CSc1ccccc1CNCCc1ccc(C#N)cc1O] | Cpd #109 | 5.1 | 102 | 3140 | 95 | 253 | 114 |
| 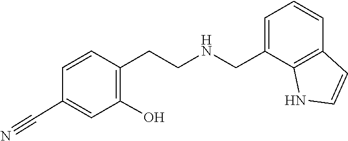 [SMILES: N#Cc1ccc(CCNCc2cccc3cc[nH]c23)c(O)c1] | Cpd #71 | 1140 | 59 | Inactive | | 10700 | 92 |
| 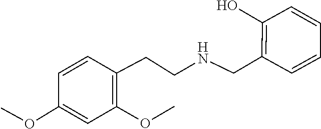 [SMILES: COc1ccc(CCNCc2ccccc2O)c(OC)c1] | Cpd #112 | 2.3 | 95 | Inactive | | 7.2 | 109 |
| 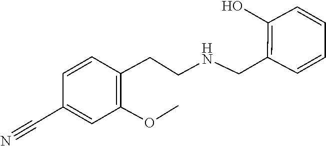 [SMILES: COc1cc(C#N)ccc1CCNCc1ccccc1O] | Cpd #113 | 4.9 | 90 | 1286 | 34 | 30.3 | 102 |
| 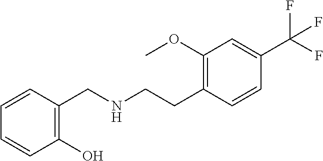 [SMILES: COc1cc(C(F)(F)F)ccc1CCNCc1ccccc1O] | Cpd #93 | 1.17 | 91 | 96.2 | 52 | 1.3 | 103 |
| 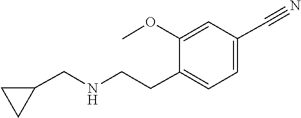 [SMILES: COc1cc(C#N)ccc1CCNCC1CC1] | Cpd #133 | 10740 | 64 | 0.07 | −6 | 34440 | 71 |
| 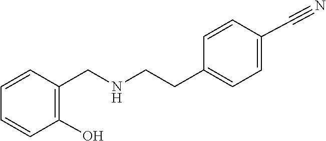 [SMILES: N#Cc1ccc(CCNCc2ccccc2O)cc1] | Cpd #114 | 154 | 88 | 6490 | 11 | 322 | 92 |
| 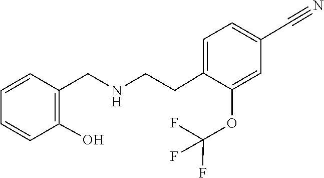 [SMILES: N#Cc1ccc(CCNCc2ccccc2O)c(OC(F)(F)F)c1] | Cpd #115 | 211.9 | 94 | Inactive | | 379.7 | 104 |
| 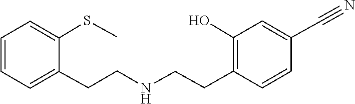 [SMILES: CSc1ccccc1CCNCCc1ccc(C#N)cc1O] | Cpd #134 | 384 | 67 | 620 | 85 | 160 | 87 |
| 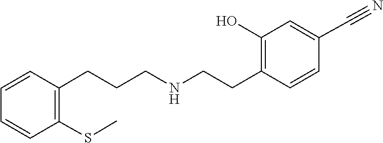 [SMILES: CSc1ccccc1CCCNCCc1ccc(C#N)cc1O] | Cpd #135 | 423 | 51 | 502 | 15 | 121 | 65 |
| 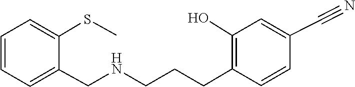 [SMILES: CSc1ccccc1CNCCCc1ccc(C#N)cc1O] | Cpd #136 | 1382 | 55 | 7330 | 49 | 412 | 43 |
| 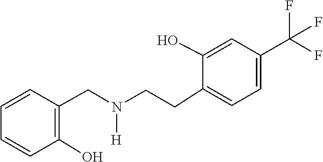 [SMILES: Oc1cc(C(F)(F)F)ccc1CCNCc1ccccc1O] | Cpd #116 | 1 | 89 | Inactive | | 1.7 | 100 |
| 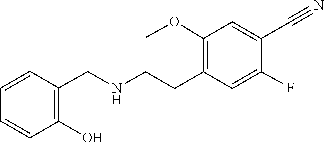 [SMILES: COc1cc(C#N)c(F)cc1CCNCc1ccccc1O] | Cpd #117 | 3.8 | 97 | 4359 | 68 | 40.8 | 115 |
| 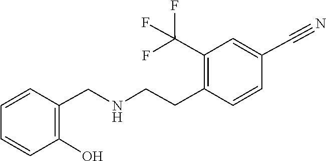 [SMILES: N#Cc1ccc(CCNCc2ccccc2O)c(C(F)(F)F)c1] | Cpd #137 | 312 | 89 | Inactive | | 56.5 | 83 |
| 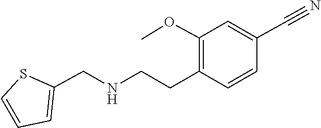 [SMILES: COc1cc(C#N)ccc1CCNCc1cccs1] | Cpd #138 | 187 | 75 | 0.03 | −2 | 198 | 102 |
| 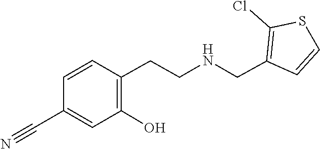 [SMILES: N#Cc1ccc(CCNCc2ccsc2Cl)c(O)c1] | Cpd #139 | 1260 | 80 | Inactive | | 4670 | 91 |
| 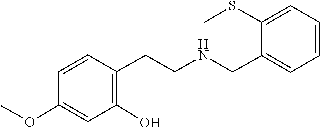 [SMILES: COc1ccc(CCNCc2ccccc2SC)c(O)c1] | Cpd #119 | 4.6 | 100 | 2543 | 108 | 113.8 | 119 |
| 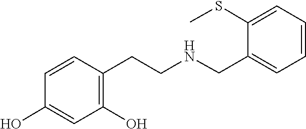 [SMILES: CSc1ccccc1CNCCc1ccc(O)cc1O] | Cpd #120 | 518 | 90 | Inactive | | Inactive | |
| 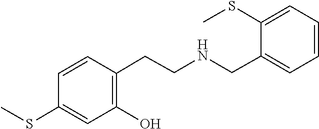 [SMILES: CSc1ccc(CCNCc2ccccc2SC)c(O)c1] | Cpd #121 | 3.6 | 94 | 175.2 | 89 | 44.1 | 110 |
| 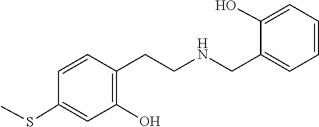 [SMILES: CSc1ccc(CCNCc2ccccc2O)c(O)c1] | Cpd #122 | 1.6 | 92 | inactive | | 3.6 | 108 |
| 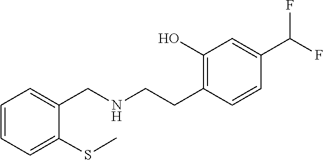 [SMILES: CSc1ccccc1CNCCc1ccc(C(F)F)cc1O] | Cpd #123 | 3.2 | 96 | 6721 | 95 | 30 | 120 |
| 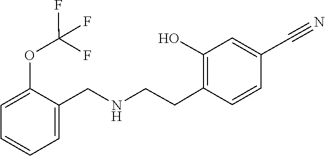 [SMILES: N#Cc1ccc(CCNCc2ccccc2OC(F)(F)F)c(O)c1] | Cpd #124 | 159 | 83 | 453.3 | 97 | 21.1 | 98 |
| 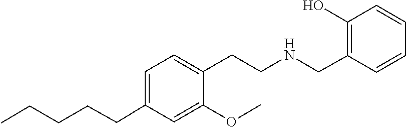 [SMILES: CCCCCc1ccc(CCNCc2ccccc2O)c(OC)c1] | Cpd #125 | 13.5 | 96 | Inactive | | 55.8 | 105 |
| 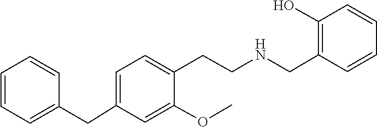 [SMILES: COc1cc(Cc2ccccc2)ccc1CCNCc1ccccc1O] | Cpd #126 | 1.2 | 99 | 0.02 | −23 | 3.8 | 111 |
| 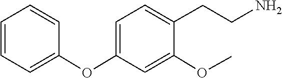 [SMILES: COc1cc(Oc2ccccc2)ccc1CCN] | Cpd #140 | 2887 | 87 | Inactive | | 795.5 | 107 |
| 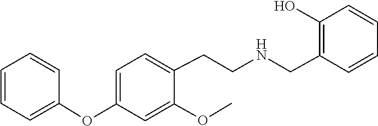 [SMILES: COc1cc(Oc2ccccc2)ccc1CCNCc1ccccc1O] | Cpd #127 | 27.1 | 100 | 3.8 | −20 | 148.2 | 104 |
| 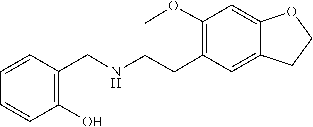 [SMILES: COc1cc2c(cc1CCNCc1ccccc1O)CCO2] | Cpd #141 | 2.3 | 96 | 78.5 | 59 | 1.6 | 107 |
| 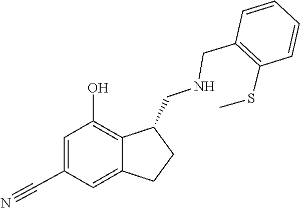 [SMILES: CSc1ccccc1CNC[C@@H]1CCc2cc(C#N)cc(O)c21] | Cpd #142 | 1958 | 68 | 18.5 | −20 | 17600 | 108 |
| 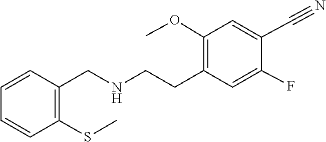 [SMILES: COc1cc(C#N)c(F)cc1CCNCc1ccccc1SC] | Cpd #128 | 7.8 | 99 | 722.7 | 109 | 114.3 | 118 |
| 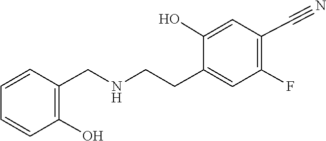 [SMILES: N#Cc1cc(O)c(CCNCc2ccccc2O)cc1F] | Cpd #129 | 7.8 | 95 | 249.3 | 62 | 67 | 105 |

| | | | | | | |
|---|---|---|---|---|---|---|
| 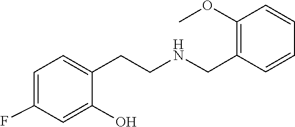 [SMILES: COc1ccccc1CNCCc1ccc(F)cc1O] | Cpd #130 | 18 | 84 | Inactive | 8.7 | 101 |
| 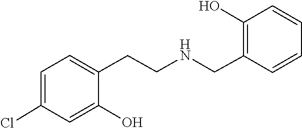 [SMILES: Oc1cc(Cl)ccc1CCNCc1ccccc1O] | Cpd #131 | 2.4 | 93 | Inactive | 1.8 | 103 |
| 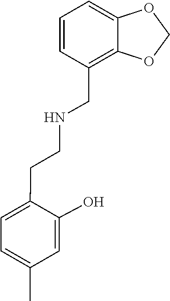 [SMILES: Cc1ccc(CCNCc2cccc3c2OCO3)c(O)c1] | Cpd #132 | 49 | 70 | Inactive | 770.1 | 84 |

Many of the compounds disclosed in this disclosure show activity in the range of up to 400 nM at the 5-HT_{2A} receptor, while showing “inactivity” at the 5-HT_{2B} receptor and possessing greater activity at the 5-HT_{2A} receptor than at the 5-HT_{2C} receptor. Many of the compounds disclosed in this disclosure selectively activate the 5-HT_{2A} receptor and show “inactivity” or limited activity at the 5-HT_{2B} receptor. For example, the compounds disclosed in this disclosure may show activity at the 5-HT_{2A} receptor in the range of less than about 1 M to about 400 nM, about 1 M to about 300 nM, about 1 M to about 200 nM, about 1 M to about 100 nM, about 1 M to about 90 nM, about 1 M to about 80 nM, about 1 M to about 70 nM, about 1 M to about 60 nM, about 1 M to about 50 nM, about 5 nM to about 95 nM, about 5 nM to about 80 nM, about 5 nM to about 65 nM, about 5 nM to about 50 nM, about 10 nM to about 90 nM, about 10 nM to about 80 nM, about 10 nM to about 70 nM, or any specific percentage therebetween. For example, the compounds disclosed in this disclosure may show selectivity for the 5-HT_{2A} receptor over the 5-HT_{2C} receptor in the following ranges: about 1 to about 100 fold, about 10 to about 100 fold, about 10 to about 90 fold, about 10 to about 80 fold, about 10 to about 70 fold, about 10 to about 60 fold, about 10 to about 50 fold, about 20 to about 100 fold, about 20 to about 80 fold, about 20 to about 60 fold, about 30 to about 90 fold, about 30 to about 60 fold, or any specific range therebetween. In an embodiment, the compound disclosed in this disclosure shows a selectivity for the 5-HT_{2A} receptor over the 5-HT_{2C} receptor of 10 to 100 fold.

Table 3 below summarizes the 5-HT receptor activity of psilocin, compound 109, and compound 93:

TABLE 3

| | Psilocin | | Compound 109 | | Compound 93 | |
|---|---|---|---|---|---|---|
| | EC₅₀, nM | Eff, % | EC₅₀, nM | Eff, % | EC₅₀, nM | Eff, % |
| 5-HT_{1A} | 46.9 | 93 | 4269 | 3 | Not active | |
| 5-HT_{1B} | 14.3 | 94 | Not active | | Not active | |
| 5-HT_{1D} | 2.2 | 102 | 378.7 | 6 | Not active | |
| 5-HT_{1E} | 10.4 | 90 | 813.8 | 12 | 70060 | 211 |
| 5-HT_{1F} | 9.3 | 93 | 582.0 | 12 | 8086 | 69 |
| 5-HT_{2A} | 14.3 | 80 | 4.8 | 99 | 0.3 | 96 |
| 5-HT_{2B} | 2.1 | 32 | 3094 | 84 | 64.2 | 50 |
| 5-HT_{2C} | 16.7 | 86 | 225.4 | 110 | 1.5 | 99 |
| 5-HT₄ | 562.6 | 81 | 0.2 | −3 | Not active | |
| 5-HT_{5A} | 349 | 48 | 12330 | −133 | Not active | |
| 5-HT₆ | 182.5 | 74 | 5394 | 18 | 0.6 | −12 |

| | | | | | |
|---|---|---|---|---|---|
| 5-HT₇ | 524.5 | 86 | Not active | 0.5 | −12 |

Phenethylamines of Formula 1 are generally known to potentially possess “hERG risk”. As a result of, many compounds disclosed in Table 1 and Table 2 were screened for the potential of “hERG risk” using pharmacological assays (e.g. Eurofins™ hERG Qube APC Assay). Compounds that exhibited strong potency at the 5-HT_{2A} receptor were further assessed on “hERG risk” based on comparative exposure/IC₅₀. A hERG IC₅₀ value that is 30-fold greater than therapeutic free plasma concentration is the threshold for deeming a compound to be of low “hERG risk”.

An important factor for hERG risk evaluation at early drug discovery stage is the hERG IC₅₀/in vitro 5-HT₂a ratio; compounds exhibiting a ratio of over 200 will exhibit a ratio of hERG IC₅₀ over estimated therapeutic plasma Cₘₐₓ of greater than 30 (which is generally indicative of low hERG risk potential at clinically relevant dose levels); compounds exhibiting a ratio between 150-200 are generally indicative of moderate hERG risk potential; compounds exhibiting a IC₅₀ (hERG) /EC₅₀ (5-HT_{2A}) ratio of less than or equal to 150 are generally indicative of high hERG risk potential. Table 4 summarizes the assessed “hERG risk” of various compounds in Tables 1 and 2.

TABLE 4

| Compound | IC₅₀, | EC₅₀ | IC₅₀ (hERG)/EC₅₀ | hERG risk |
|---|---|---|---|---|
| # | μM | BRET _{2A} | (5-HT_{2A}) ratio | potential |
| 109 | 2.9 | 4.8 | 604 | Low |
| 108 | 1.78 | 2.4 | 742 | Low |
| 27 | 2 | 4.2 | 476 | Low |
| 117 | 0.9 | 3.8 | 237 | Low |
| 93 | 0.62 | 1.2 | 517 | Low |
| 112 | 6 | 2.3 | 2609 | Low |
| 12 | 1.72 | 102 | 17 | High |
| 65 | 2 | 19.7 | 102 | High |
| 28 | 1.6 | 84 | 19 | High |
| 63 | 1.6 | 135 | 12 | High |
| 64 | 0.9 | 29.8 | 30 | High |
| 131 | 1.31 | 2.1 | 624 | Low |
| 122 | 0.79 | 1.4 | 564 | Low |
| Ref | 2.3 | 451 | 5 | High |
| Compound | | | | |
| 1a | | | | |

Various compounds disclosed herein were subjected to an in vitro SafetyScreen44™ panel for identifying off-target activity. Compounds of interest that are identified in the panel are then tested in follow up functional assays to identify if such compounds have a significant off-target risk. As summarized below in Table 5, compound 109 does not possess significant off-target activity. It is predicted that other compounds share similar characteristics with compound 109 and also lack of the off-target activity.

TABLE 5

| | SafetyScreen44 ™ | |
|---|---|---|
| Compound | panel hits | Follow up studies |
| 109 | α 1a | No significant |
| | α 2a | off-target activity |
| | Kappa (KOP) | |
| | μ (MOP) | |
| | 5-HT transporter | |
| | (SERT) | |

Additional experimentation was performed on compound 109. Table 6 summarizes, among other things, the anticipated hepatic clearance of compound 109 in various species, as specified in Table 6 and as determined by calculating the in vitro hepatocyte clearance over time and scaling such rate with such species' hepatocyte count and the anticipated liver blood flow rate in such species.

TABLE 6

| | | In vitro | Scale- | Hepatic | |
|---|---|---|---|---|---|
| | In vitro | Clᵢₙₜ | up Clᵢₙₜ | Extraction | Predicted |
| | T_{1/2} | (μL/min/ | (mL/min/ | Ratio | hepatic CL |
| Species | (min) | 10⁶ cells) | kg) | (E) | (mL/min/kg) |
| Human | 2.28 | 608.72 | 1548.76 | 0.99 | 20.43 |
| Monkey | 2.78 | 499.05 | 1796.57 | 0.98 | 42.57 |
| Dog | 6.96 | 199.06 | 1369.52 | 0.98 | 30.22 |
| Rat | 5.93 | 233.71 | 1093.74 | 0.95 | 52.55 |
| Mouse | <2.26 | >614.02 | >7253.14 | >0.99 | >88.90 |

Table 7a and Table 7b summarize, among other things, the observed plasma and Brain P K properties in animals, and the observed plasma and Brain P K properties predicted in humans, for compound 109 in Table 7a and compound 93 for Table 7b:

TABLE 7a

| | | | | T_{1/2} | IM | Brain |
|---|---|---|---|---|---|---|
| | BW | Clₜₒₜ | Vz | Vz | f | Plasma |
| Species | (kg) | [mL/h/Kg] | [mL/Kg] | (h) | (%) | AUC Ratio |
| mice | 0.029 | 17170.2 | 14040 | 0.6 | 144.5 | 8.39 |
| rat | 0.207 | 3204 | 9370 | 2.1 | 92.8 | 2.18 |
| Dog | 8.240 | 2382 | 4330 | 1.3 | 120.0 | No Data |
| | | | | | | Provided |
| Human | 70.0 | 971.1 | 2775.3 | 2.0 | 100.0 | 2.14 |
| (predicted) | | | | | | |

TABLE 7b

| | BW | Clₜₒₜ | Vz | T_{1/2} | Brain/plasma |
|---|---|---|---|---|---|
| Species | (kg) | [mL/h/Kg] | [mL/Kg] | Vz(h) | Ratio |
| mice | 0.0265 | 11795 | 15112 | 0.9 | 15.6 |
| rat | 0.232 | 7622 | 15192 | 1.38 | 19.6 |
| Dog | 9.22 | 3188 | 7703 | 1.66 | No Data |
| | | | | | Provided |
| Human | 70 | 2271 | 6391.8 | 1.95 | 9.55 |
| (predicted) | | | | | |

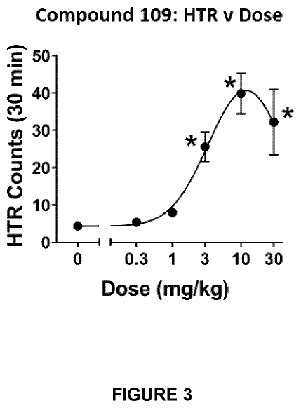

HTR was assessed using a head-mounted neodymium magnet and a magnetometer detection coil, as described in Halberstadt et al., Psychopharmacology (Berl.), 2013, 227(4): 727-739. Table 8 below summarizes the ED₅₀ in head twitch in mice of various compounds disclosed herein as compared to psilocybin. FIG. 3 illustrates the HTR relative to dose for compound 109:

TABLE 8

| Cpd # | ED₅₀ in Head Twitch |
|---|---|
| Psilocybin | 0.38 mg/kg |
| 112 | 0.73 mg/kg |
| 109 | 2.69 mg/kg |
| 108 | 7.23 mg/kg |
| 93 | 0.28 mg/kg |
| 27 | 3.00 mg/kg |
| 117 | 2.09 mg/kg |
| 131 | 1.24 mg/kg |

The experimental data summarized in Tables 6-8 demonstrate that compound 109 exhibits high brain penetration, but less central hallucinogenic (or psychedelic) effects than traditional psychedelic compounds such as psilocybin.

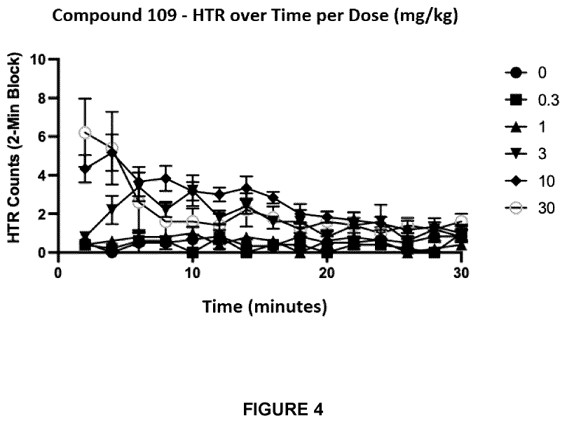

Referring to FIG. 4, the short-lasting (less than 30 minutes) psychedelic effect of compound 109 is further illustrated. Per FIG. 4, HTR over time per different doses of compound 109 (as measured in mg/kg) is shown. Similar short lasting (less than 30 minutes) psychedelic effects (as suggested by HTR data) is observed in other compounds disclosed in this disclosure. Compound 109 and other like compounds also exhibit long lasting antidepressant effects (neuroplasticity modulation). In contrast, many psychedelic compounds known in the art, liked psilocybin, have long lasting (more than 30 minutes) psychedelic effects; such long lasting psychedelic effects are undesirable from a medical treatment perspective.

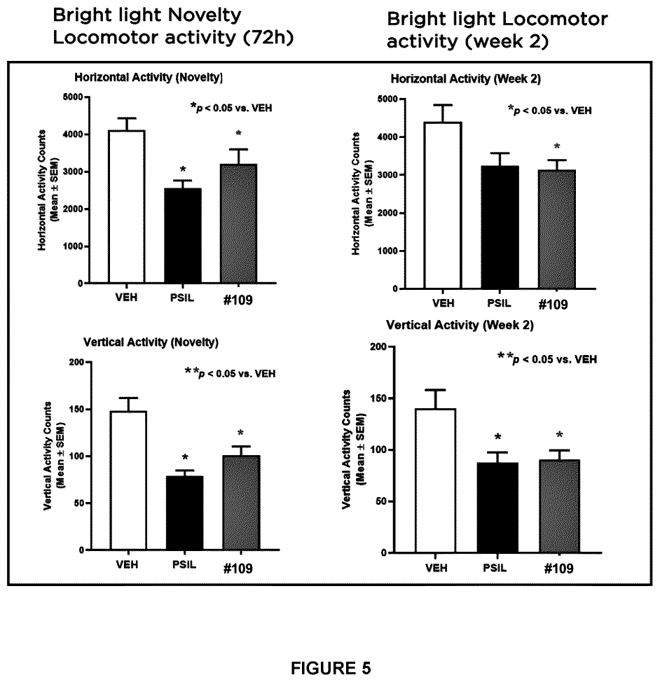

Additional experimentation was also done on compound 109. Namely, an intraperitoneal dose of up to 30 mg/kg of compound 109 and various reference compounds were delivered into healthy rat subjects and surgery induced abnormal rat subjects (olfactory bulbectomized rats). With regard to compound 109, there is no suggestion that an intraperitoneal dose of up to 30 mg/kg of such compound affects the normal locomotor activity of healthy rat subjects. However, a single dose of such intraperitoneal dose showed significant and long lasting inhibition of abnormal behavioral activity in the surgery induced abnormal rat subjects, in contrast to its short plasma t_{1/2} of less than an hour in healthy rat subjects. Without being bound by theory, it is believed that these observations are a result of long lasting neuroplasticity effects of compound 109 on surgery induced abnormal rat subjects. FIG. 5 summarizes the results of this experiment. Using psilocybin as a comparative psychedelic, the results of this experiment support the notion that compound 109 provides sustainable acute antidepressant effects. Note that “VEH” refers to vehicle, and “PSIL” refers to psilocybin, in FIG. 5.

Compound 93 also exhibited high clearance in in vitro tests. The results of this experiment are summarized in Table 9 below:

TABLE 9

| | | In vitro | Scale- | |
|---|---|---|---|---|
| | In vitro | Clᵢₙₜ | up Clᵢₙₜ | Predicted |
| | T_{1/2} | (μL/min/ | (mL/min/ | hepatic CL |
| Species | (min) | 10⁶ cells) | kg) | (mL/min/kg) |
| Human | 25.28 | 54.83 | 139.52 | 18.03 |
| Monkey | 2.97 | 466.96 | 1681.07 | 42.50 |
| dog | 4.11 | 337.46 | 2321.69 | 30.49 |
| Rat | >2.26 | <614.02 | <2873.63 | <54.16 |
| Mouse | >2.26 | <614.02 | <7253.14 | <88.90 |

### Methods of Chemical Synthesis

Non-limiting examples of procedures for preparing the compounds described herein are provided below.

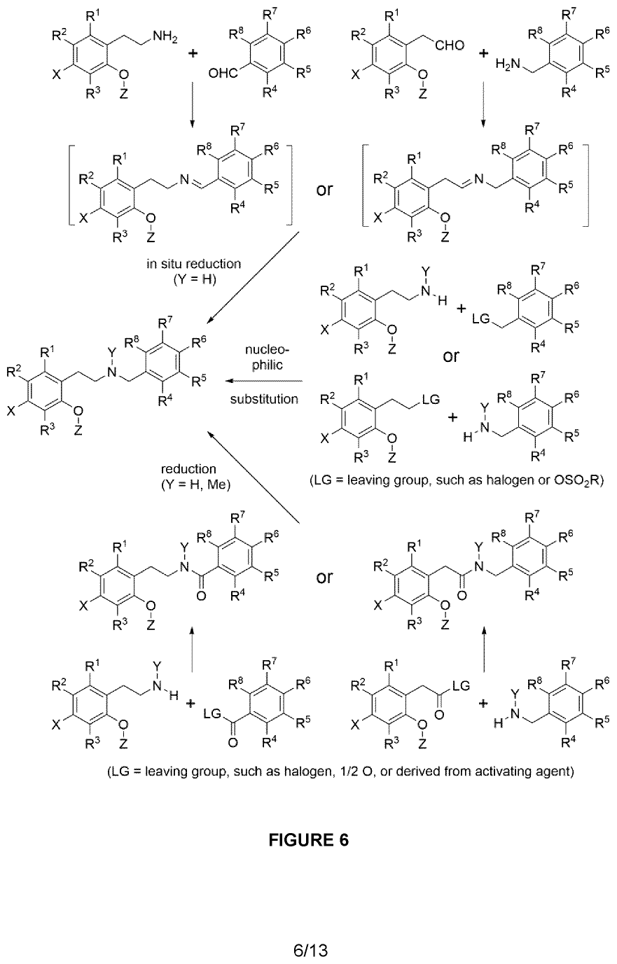

N-Benzylphenethylamines can be prepared by N-benzylation of phenethylamines or N-phenethylation of benzylamines. These reactions may be carried out in a manner that leads directly to products in the oxidation state of amines using, for example, halides or sulfonates as alkylating agents. The amine nitrogen may be present in free form; or to avoid the common side-reactions of dialkylation and quaternization, the amine nitrogen may carry a protecting group that is removed at a later stage of the synthesis. The reactions may also be carried out using electrophiles at a higher oxidation state to obtain products such as imines or carboxamides that need to be reduced in situ or in a subsequent step to arrive at the desired amines. For example, a commonly employed protocol to effect the reductive alkylation of a primary amine consists of treating the amine with an aldehyde in the presence of a mild reducing agent, such as sodium borohydride, sodium cyanoborohydride, or sodium triacetoxyborohydride. Dialkylation and direct reduction of the aldehyde prior to imine formation represent frequently observed side reactions. Amines also readily react with acylating agents, such as acyl halides, carboxylic acid anhydrides, or free carboxylic acids in the presence of condensating agents, of which numerous representatives are known from research in the field of peptide synthesis, to form carboxamides. Unlike the often hydrolytically sensitive imines, carboxamides are stable compounds that are isolated and then reduced to amines in a subsequent step. This reduction process requires either strong reducing agents, such as lithium aluminum hydride, sodium bis(2-methoxyethoxy)aluminum hydride (Red-Al®), or borane, which are usually incompatible with the nitrile function that is present in many of the target compounds; or the carboxamide needs to be activated by formation of a mixed imidic acid anhydride upon treatment with a strong electrophile. Employing triflic anhydride in that role, carboxamides are reduced to amines without affecting nitriles and a variety of other reducible functional groups by sodium borohydride (Huang, P.-Q.; Geng, H. Org. Chem. Front. 2015, 2, 150) or by the mild hydride donor, Hantzsch ester (Pelletier, G.; Bechara, W. S.; Charette, A. B. J. Am. Chem. Soc. 2010, 132, 12817). For a=b=c=H, the general synthetic scheme for the present target compounds of Formula I is shown in FIG. 6.

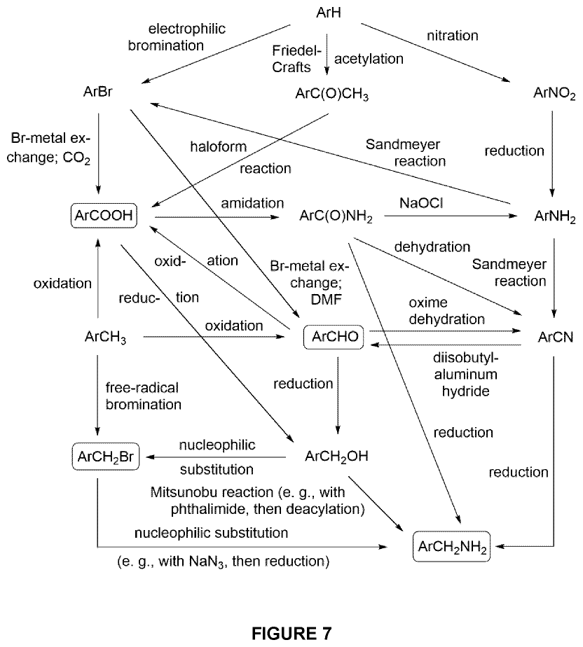

The building blocks for the benzyl portion of phenethylamine can, among other possible approaches, be prepared by standard functional group transformations as shown (without details in the scheme) in FIG. 7; in such Figure, Ar is a phenyl group bearing the substituents R⁴, R⁵, R⁶, R⁷, and R⁸ as in Formula I.

The same and additional functional group transformations can be enlisted to obtain commercially unavailable patterns of the substituents R¹, R², R³, R⁴, R⁵, R⁶, R⁷, and R⁸. Halogen substituents, including F, can be accessed by halodecarboxylation of carboxylic acids (Varenikov, A.; Shapiro, E.; Gandelman, M. Chem. Rev. 2021, 121, 412). Aromatic fluorides can also be obtained from numerous other aromatic starting materials (Campbell, M. G.; Ritter, T. Chem. Rev. 2015, 115, 612), for example from aromatic amines through the Balz-Schiemann reaction, or from other halides by halogen exchange. Transition-metal-mediated halogen exchange is applicable to a variety of combinations of the leaving and incoming halogen (Evano, G.; Nitelet, A.; Thilmany, P.; Dewez, D. F. Front. Chem. 2018, 6, article No. 114). Aryl triflates, readily available derivatives of phenols, also participate in this type of reaction (Sather, A. C.; Lee, H. G.; De La Rosa, V. Y.; Yang, Y.; Müller, P.; Buchwald, S. L. J. Am. Chem. Soc. 2015, 137, 13433). Trifluoromethylated aromatics can be synthesized, for example, from carboxylic acids with SF₄; from amines by Sandmeyer reaction with sodium trifluoromethanesulfonate (Hong, J.; Wang, G.; Huo, L.; Zheng, C. Chin. J. Chem. 2017, 35, 761) orTogni's reagent II (1-trifluoromethyl-1,2-benziodoxol-3(1H)-one; Hong, J.; Huo, L.; Yang, Y.; Wang, G.; Zheng, C. Chem. Select 2017, 2, 3716); or from aryl halides by transition-metal-catalyzed nucleophilic trifluoromethylation (Roy, S.; Gregg, B. T.; Gribble, G. W.; Le, V.-D.; Roy, S. Tetrahedron 2011, 67, 2161). Difluoromethyl substituents are frequently generated from aldehydes by deoxofluorination. The group of reagents capable of performing this transformation has recently been enlarged by the combination of Selectfluor® and diphenyl sulfide (He, G.; Xiao, X.; Jin, H.-Z.; Lin, J.-H.; Zhong, T.; Zheng, X.; Xiao, J. C. Tetrahedron 2021, 83, article No. 131963). (Trifluoromethoxy) arenes can be obtained from phenols by reaction of their derived xanthates with the commercial fluorinating agent, XtalFluor-E® (diethylaminodifluorosulfinium tetrafluoroborate (Yoritate, M.; Londregan, A. T.; Lian, Y.; Hartwig, J. F. J. Org. Chem. 2019, 84, 15767). Aromatic amines are transformed to (trifluoromethoxy) arenes through action of AgOCF₃ on their derived diazonium salts (Org. Lett. 2019, 21, 8003). The trifluoromethylthio group can be introduced into aromatics by displacement of other heteroatoms with the SCF₃ moiety, or by electrophilic substitution of hydrogen in electron-rich aromatics (Barata-Vallejo, S.; Bonesi, S.; Postigo, A. Org. Biomol. Chem. 2016, 14, 7150). (Trifluoromethylthio) arenes are also obtained from arylthiols by reaction with electrophilic or free-radical trifluoromethylating agents, such as S-(trifluoromethyl)sulfonium salts or CF₃ halides (review together with other methods: Xu, X.-H.; Matsuzaki, K.; Shibata, N. Chem. Rev. 2015, 115, 731). (Difluoromethoxy)—and (difluoromethylthio) arenes are obtained from phenols and arenethiols, respectively, and various sources of difluorocarbene, in a recent example, an S-(difluoromethyl)diarylsulfonium salt (Liu, G.-K.; Qin, W.-B.; Li, X.; Lin, L.-T.; Wong, H. N. C. J. Org. Chem. 2019, 84, 15948).

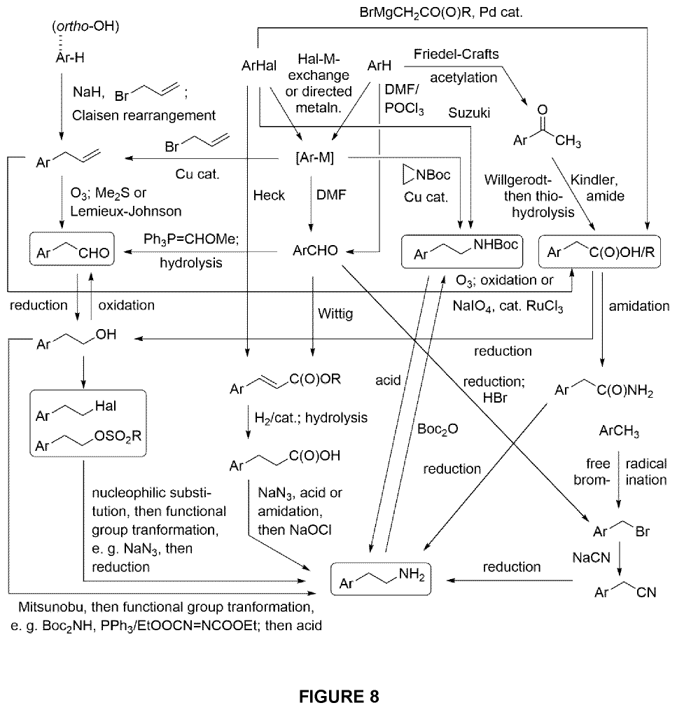

Building blocks for the phenethyl portion can, among other possible approaches, be prepared by standard functional group transformations as generally shown in the scheme depicted in FIG. 8, wherein Ar is a phenyl group bearing the substituents R¹, R², and R³ as in Formula 1.

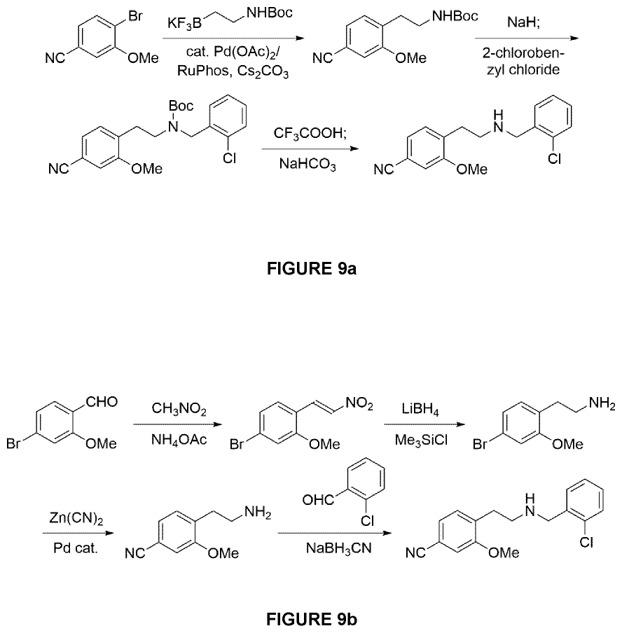

As an example for the synthetic methods of the preceding paragraphs, two syntheses of the compound 12 are depicted in FIGS. 9a and 9b.

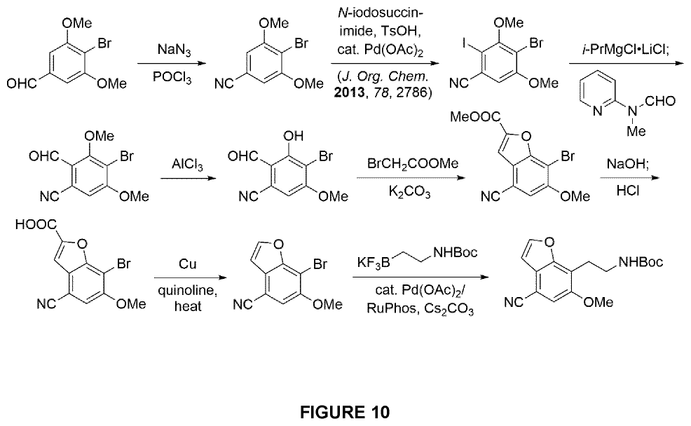

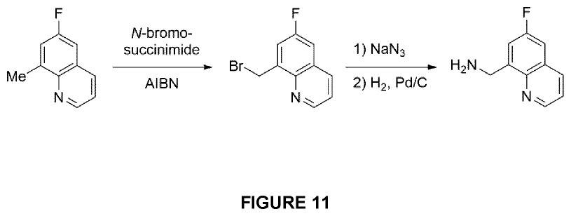

Various heterocycles annulated to either benzene ring are encountered in certain compounds of this disclosure, resulting from the presence of linkages between R¹ and R², R³ and Z, and/or R⁴ and R⁵. As examples, syntheses of the phenethylamine building block for compound 32 (with a linkage between R¹ and R²) and of the benzylamine building block for compound 76 (with a linkage between R⁴ and R⁵) are depicted in FIGS. 10 and 11 respectively.

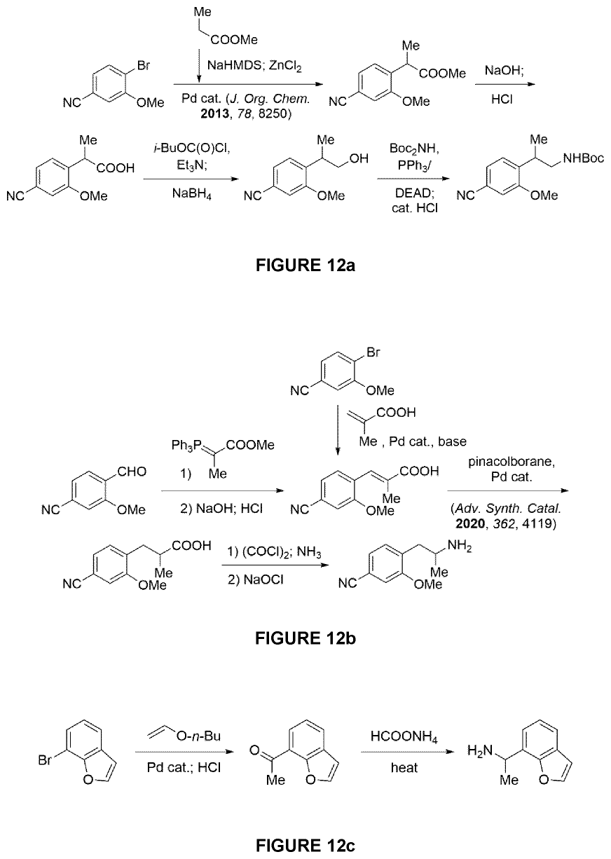

FIGS. 12a, 12b, and 12c respectively depict the syntheses of building blocks in which a, b, or c is a methyl group.

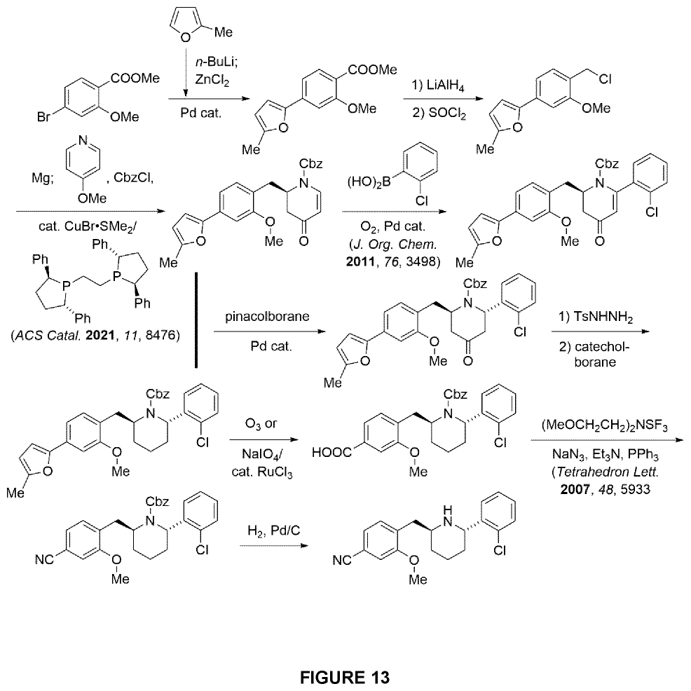

FIG. 13 shows a scheme for the synthesis of one of four stereoisomers of compound 86, in which b and c are connected to form a ring, having the absolute and relative stereochemistry shown.

Deuterium may be incorporated into the present compounds in various ways, using deuterated versions of reagents and building blocks under the same or similar conditions as those employed for their counterparts with natural hydrogen isotope composition. The reduction of carboxamides with commercially available LiAID₄ or BD₃-THF complex results in amines deuterated on the methylene group that originates from the amides' carbonyl group. Similarly, reductive aminations/alkylations can be effected with NaBD₄ and NaBD₃CN. Aromatics and heteroaromatics can also be deuterated by reaction with an excess of D₂O in the presence of a heterogeneous transition metal catalyst (Sawama, Y.; Park, K.; Yamada, T.; Sajiki, H. Chem. Pharm. Bull. 2018, 66, 21-28). Deuteration of specific positions in aromatic or heteroaromatic rings is achievable by halogen-metal exchange reactions on compounds that bear a halogen atom (typically Br or 1) at the position to be deuterated, followed by quenching of the (hetero-)arylmetal intermediate with a deuterating agent such as D₂O or CH₃OD; or by free-radical deuterodehalogenation of the same precursors with Bu₃SnD and a radical starter such as azobis(isobutyronitrile) or dibenzoyl peroxide; or by reaction of the same precursors with a deuteride source such as Bu₃SnD or formic acid-d₂ and a transition metal catalyst.

### Methods of Use

Compounds described herein are believed to be useful in the treatment of depression (including major depressive disorder), drug resistant depression, psychotic depression, and PTSD, as well as to serve as stimulants, anorectics, decongestants, bronchodilators, and as psychedelics. In addition, the compounds disclosed herein are also believed to be useful in the treatment of addiction including alcoholism, tobacco addiction, cocaine addiction, and opioid addiction, pain indications including neuropathic pain, pain from chemotherapy associated neuropathy, phantom limb pain and fibromyalgia, inflammation (including chronic and acute), eating disorders including anorexia, autism, cluster headaches, migraines, dementia including Alzheimer's dementia, Parkinson's disease dementia, and Lewy body dementia, emotional distress associated with cancer, Fragile-X syndrome, autism spectrum disorder, bipolar disease, obsessive compulsive disease, Rett syndrome, and other CNS disorders.

Substituted phenethylamines make up a group of phenethylamine derivatives that contain phenethylamine as a “backbone”; in other words, this chemical class includes derivatives that are formed by replacing one or more of the hydrogen atoms in the phenethylamine scaffolding with other substituents. The class of substituted phenethylamines includes all substituted amphetamines, and substituted methylenedioxyphenethylamines (e.g., MDMA), and contains many drugs which act as empathogens, stimulants, psychedelics, anorectics, bronchodilators, decongestants, and/or antidepressants, inter alia.

Another potential use of these analogs is in the treatment of seizure disorders, including but not limited to infantile seizure disorders such as Dravet syndrome (Sourbon, J. et al. “Serotonergic Modulation as Effective Treatment for Dravet Syndrome in a Zebrafish Mutant Model”, ACS Chem. Neurosci. 2016, 7, 588-598).

Another potential use of these analogs is in the treatment of compulsive disorders, anxiety disorders, fear disorders and aggressiveness in animals; non-limiting examples of animals include dogs, cats, pigs, mice and rats. Another potential use of these analogs is in inducing contraction of the ureter in animals; non-limiting examples of animals include dogs, cats, pigs, mice and rats.

The compounds described herein are believed to be safer than psilocybin, given that they lack at least some of the undesirable characteristics of 5-HT_{2B}-agonist related activities.

### Methods for Administration

As contemplated herein, a therapeutically effective amount of a compound described herein is administered to a subject in need thereof. Whether such treatment is indicated depends on the subject case, and is further subject to medical assessment (diagnosis) that takes into consideration signs, symptoms, and/or malfunctions that are present, the risks of developing particular signs, symptoms and/or malfunctions, and other factors.

As contemplated herein, a compound described herein may be administered by any suitable route known in the art. Such routes include, but are not limited to, oral, buccal, inhalation, topical, sublingual, rectal, vaginal, intracisternal or intrathecal through lumbar puncture, transurethral, nasal, percutaneous, transdermal, and parenteral administration (including intravenous, intramuscular, subcutaneous, intracoronary, intradermal, intramammary, intraperitoneal, intraarticular, intrathecal, retrobulbar, intrapulmonary injection and/or surgical implantation at a particular site). Parenteral administration may be accomplished using a needle and syringe or using a high pressure technique.

Pharmaceutical compositions include those wherein a compound described herein is present in a sufficient amount to be administered in an effective amount to achieve its intended purpose. The exact formulation, route of administration, and dosage is determined by a qualified medical practitioner in view of the diagnosed condition or disease. Dosage amount and interval can be adjusted individually to provide levels of a compound described herein that is sufficient to maintain the desired therapeutic effects. It is possible that the compound described herein may only require infrequent administration (e.g. monthly, as opposed to daily) to achieve the desired therapeutic effect.

As contemplated herein, a therapeutically effective amount of a compound described herein adapted for use in therapy varies with the nature of the condition being treated, the length of time that activity is desired, and the age and the condition of the patient, and ultimately is determined by the attendant physician. Dosage amounts and intervals can be adjusted individually to provide plasma levels of the compound that are sufficient to maintain the desired therapeutic effects. The desired dose conveniently may be administered in a single dose, or as multiple doses administered at appropriate intervals, for example as one, two, three, four, or more subdoses per day. Multiple doses often may be desired or required.

As contemplated herein, the compounds described herein may be administered in admixture with a pharmaceutical carrier selected with regard to the intended route of administration and standard pharmaceutical practice. Pharmaceutical compositions for use in accordance with the compounds described herein are formulated in a conventional manner using one or more physiologically acceptable carriers comprising excipients and auxiliaries that facilitate processing of the compounds described herein.

Water is a preferred carrier when a compound described herein is administered intravenously. Saline solutions and aqueous dextrose and glycerol solutions may also be used as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The present compositions, if desired, can also contain minor amounts of wetting or emulsifying agents, or pH buffering agents.

These pharmaceutical compositions may be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of a compound described herein is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a compound described herein. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a compound described herein.

When a therapeutically effective amount of a compound described herein described herein is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains an isotonic vehicle. A compound described herein can be infused with other fluids over a 10-30 minute span or over several hours.

The compounds described herein may be readily combined with pharmaceutically acceptable carriers well-known in the art. Such carriers enable the active agents to be formulated as tablets, pills, dragees, capsules, liquids, gels, syrups, slurries, suspensions and the like, for oral ingestion by a patient to be treated.

Pharmaceutical preparations for oral use can be obtained by adding a compound described herein to a solid excipient, with or without grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

A compound described herein may be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a compounds described herein can be prepared as appropriate oily injection suspensions. Suitable lipophilic solvents or vehicles include fatty oils or synthetic fatty acid esters. Aqueous injection suspensions can contain substances which increase the viscosity of the suspension.

In some embodiments, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

A compound described herein also may be formulated in rectal compositions, such as suppositories or retention enemas, e.g., containing conventional suppository bases. In addition to the formulations described previously, a compound described herein also can be formulated as a depot preparation. Such long-acting formulations can be administered by implantation (for example, subcutaneously or intramuscularly) or by intramuscular injection. Thus, for example, a compound described herein may be formulated with suitable polymeric or hydrophobic materials (for example, as an emulsion in an acceptable oil) or ion exchange resins.

A compound described herein may be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. The compounds described herein also may be injected parenterally, for example, intravenously, intramuscularly, subcutaneously, or intracoronarily. For parenteral administration, the compounds described herein may be best used in the form of a sterile aqueous solution which can contain other substances, for example, salts or monosaccharides, such as mannitol or glucose, to make the solution isotonic with blood. At least in some embodiments, compounds described herein are psilocybin analogs.

### General

It is contemplated that any part of any aspect or embodiment discussed in this specification may be implemented or combined with any part of any other aspect or embodiment discussed in this specification. While particular embodiments have been described in the foregoing, it is to be understood that other embodiments are possible and are intended to be included herein. It will be clear to any person skilled in the art that modification of and adjustment to the foregoing embodiments, not shown, is possible.

Unless defined otherwise, all technical and scientific terms used herein have the same meaning as is commonly understood by one of ordinary skill in the art to which the content of this disclosure belongs. In addition, any citation of references herein is not to be construed nor considered as an admission that such references are prior art that are relevant to the content of this disclosure.

The scope of the claims should not be limited by the example embodiments set forth herein, but should be given the broadest interpretation consistent with the description as a whole.

1. A chemical compound of Formula 1, or isotopologue or pharmaceutically acceptable salt thereof: 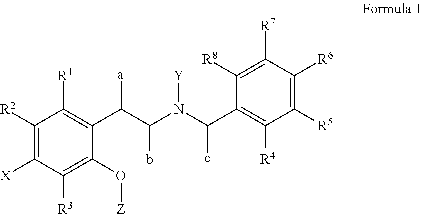
wherein:
R¹: (i) is selected from the group consisting of H, C₁-C₄ alkyl, substituted C₁-C₄ alkyl, cyclopropyl, cyclobutyl, cyclopropylmethyl, 2-oxetanyl, 3-oxetanyl, OH, C₁-C₄ alkoxy, substituted C₁-C₄ alkoxy, C₁-C₄ alkylthio, substituted C₁-C₄ alkylthio, and halogen, if each of R² and R³ is independently selected from H, CH₃, or halogen; or (ii) together with R² form an alkanediyl, alkenediyl, heteroalkanediyl, or heteroalkenediyl moiety; or (iii) together with b form an alkanediyl, alkenediyl, or heteroalkanediyl moiety;
R²: (i) is selected from the group consisting of H, C₁-C₄ alkyl, substituted C₁-C₄ alkyl, cyclopropyl, cyclobutyl, cyclopropylmethyl, 2-oxetanyl, 3-oxetanyl, OH, C₁-C₄ alkoxy, substituted C₁-C₄ alkoxy, C₁-C₄ alkylthio, substituted C₁-C₄ alkylthio, and halogen, if each of R¹ and R³ is independently selected from H, CH₃, or halogen; or (ii) together with R¹ form an alkanediyl, alkenediyl, heteroalkanediyl, or heteroalkenediyl moiety;
R³: (i) is selected from the group consisting of H, C₁-C₄ alkyl, substituted C₁-C₄ alkyl, cyclopropyl, cyclobutyl, cyclopropylmethyl, 2-oxetanyl, 3-oxetanyl, OH, C₁-C₄ alkoxy, substituted C₁-C₄ alkoxy, C₁-C₄ alkylthio, substituted C₁-C₄ alkylthio, and halogen, if each of R¹ and R² is independently selected from H, CH₃, or halogen; or (ii) together with Z form an alkanediyl, alkenediyl, heteroalkanediyl, or heteroalkenediyl moiety;
wherein:
one of R⁴, R⁵, R⁶, R⁷, and R⁸ is selected from the group consisting of R⁹, OR⁹, SR⁹, S(O)R⁹, S(O)₂R⁹, N(R⁹)C(O)R⁹, N(R⁹)C(O)OR⁹, N(R⁹)C(O)N(H)R⁹, N(R⁹)C(O)N(C₁-C₆ alkyl)R⁹, N(R⁹)S(O)₂R⁹, CH₂OR⁹, CH₂SR⁹, CH₂S(O)R⁹, CH₂S(O)₂R⁹, CH₂N(R⁹)C(O)R⁹, CH₂N(R⁹)C(O)OR⁹, CH₂N(R⁹)C(O)N(H)R⁹, CH₂N(R⁹)C(O)N(C₁-C₆ alkyl)R⁹, CH₂N(R⁹)S(O)₂R⁹, CH═NOR⁹, C(C₁-C₆ alkyl)═NOR⁹, C(O)OR⁹, C(O)N(H)R⁹, C(O)N(C₁-C₆ alkyl)R⁹, CH₂C(O)OR⁹, CH₂C(O)N(H)R⁹, CH₂C(O)N(C₁-C₆ alkyl)R⁹, CN, and halogen, a second one of R⁴, R⁵, R⁶, R⁷ or R⁸ is selected from the group consisting of H, C₁-C₄ alkyl, CHF₂, CF₃, OH, OCH₃, OC₂H₅, OCHF₂, OCF₃, and halogen, and the remainder of R⁴, R⁵, R⁶, R⁷, and R⁸ are each independently H, CH₃, or halogen;
or if R⁴ and R⁵ together form an alkanediyl, alkenediyl, heteroalkanediyl, or heteroalkenediyl moiety, then each of R⁶, R⁷, and R⁸ is independently selected from the group consisting of H, CH₃, and halogen;
or if R⁴ and b together form an alkanediyl, alkenediyl, or heteroalkanediyl moiety, then one of R⁵, R⁶, R⁷, and R⁸ is selected from the group consisting of H, C₁-C₄ alkyl, CHF₂, CF₃, OH, OCH₃, OC₂H₅, OCHF₂, OCF₃, and halogen, and the remainder of R⁵, R⁶, R⁷, and R⁸ are each independently selected from the group consisting of H, CH₃, and halogen;
or if R⁴ and together form an alkanediyl, alkenediyl, or heteroalkanediyl moiety, then one of R⁵, R⁶, R⁷, and R⁸ is selected from the group consisting of H, C₁-C₄ alkyl, CHF₂, CF₃, OH, OCH₃, OC₂H₅, OCHF₂, OCF₃, CN, and halogen, and the remainder of R⁵, R⁶, R⁷, and R⁸ are each independently selected from the group consisting of H, CH₃, and halogen;
wherein:
X is selected from the group consisting of CN, C(O)NH₂, C(O)N(H)R⁹, C(O)N(C₁-C₆ alkyl)R⁹, C(O)(C₄-C₆ heterocyclyl), CHF₂, CF₃, OH, O(C₁-C₆ alkyl), OCHF₂, OCF₃, S(C₁-C₆ alkyl), SCF₃, SCHF₂, F, C, aryl, and heteroaryl generally;
Y is selected from the group consisting of H, CH₃, C(O)R⁹, CH₂OC(O)R⁹, and C(O)OR⁹; Z: (i) is selected from the group consisting of H, C₁-C₁₀ alkyl, substituted C₁-C₁₀ alkyl, C₁-C₁₀ heteroalkyl, C₂-C₁₀ alkenyl, C₂-C₁₀ heteroalkenyl, C₂-C₁₀ alkynyl, C₃-C₆ cycloalkyl, (C₃-C₆ cycloalkyl)(C₁-C₃ alkyl), (C₃-C₆ cycloalkyl)(C₁-C₃ heteroalkyl), C₄-C₆ heterocyclyl, (C₄-C₆ heterocyclyl)(C₁-C₃ alkyl), (C₄-C₆ heterocyclyl)(C₁-C₃ heteroalkyl), aryl(C₁-C₃ alkyl), aryl(C₁-C₃ heteroalkyl), heteroaryl(C₁-C₃ alkyl), heteroaryl(C₁-C₃ heteroalkyl), C(O)R⁹, C(O)OR⁹, CH₂OC(O)R⁹, C(O)NH₂, C(O)N(H)R⁹, C(O)N(C₁-C₆ alkyl)R⁹, and C(O)(C₄-C₆ heterocyclyl); or (ii) together with R³ form an alkanediyl, alkenediyl, heteroalkanediyl, or heteroalkenediyl moiety;
wherein two of a, b, and c are each independently selected from the group consisting of H, CH₃, and C₂H₅, while the third of a, b, and c is H, but if c and R⁴ together form an alkanediyl, alkenediyl, or heteroalkanediyl moiety then a and b are each independently selected from H or CH₃; and
wherein R⁹ is, independently for each occurrence, selected from the group consisting of H, C₁-C₆ alkyl, C₁-C₆ heteroalkyl, substituted C₁-C₆ alkyl, substituted C₁-C₆ heteroalkyl, C₂-C₆ alkenyl, C₂-C₆ heteroalkenyl, C₂-C₆ alkynyl, C₃-C₆ cycloalkyl, (C₃-C₆ cycloalkyl)(C₁-C₆ alkyl), (C₃-C₆ cycloalkyl)(C₁-C₆ heteroalkyl), C₃-C₆ heterocyclyl, (C₃-C₆ heterocyclyl)(C₁-C₆ alkyl), (C₃-C₆ heterocyclyl)(C₁-C₆ heteroalkyl), aryl, aryl(C₁-C₆ alkyl), aryl(C₁-C₆ heteroalkyl), heteroaryl, heteroaryl(C₁-C₆ alkyl), and heteroaryl(C₁-C₆ heteroalkyl).

2. The compound as claimed in claim 1, or isotopologue or pharmaceutically acceptable salt thereof, wherein: (i) X is selected from the group consisting of CN, OH, O(C₁-C₆ alkyl), OCHF₂, OCF₃, S(C₁-C₆ alkyl), SCF₃, SCHF₂, F, Cl, aryl, and heteroaryl; and (ii) Z is selected from the group consisting of H and C₁-C₁₀ alkyl.

3. The compound as claimed in claim 2, or isotopologue or pharmaceutically acceptable salt thereof, wherein X is selected from the group consisting of CN, OH, OCH₃, F, C, CF₃, SCH₃, an oxazole, a pyrimidine, and an isoxazole.

4. The compound as claimed in claim 2, or isotopologue or pharmaceutically acceptable salt thereof, wherein R⁴ is selected from the group consisting of H, F, Cl, OH, OR⁹, and SCH₃.

5. The compound as claimed in claim 4, or isotopologue or pharmaceutically acceptable salt thereof, wherein: (i) X is CN; (ii) Z is H or CH₃; and (iii) R⁴ is SCH₃.

6. The compound as claimed in claim 5, or isotopologue or pharmaceutically acceptable salt thereof, wherein R¹, R², R³, a, b, y, c, R⁵, R⁶, R⁷, and R⁸ are H.

7. The compound as claimed in claim 5, or isotopologue or pharmaceutically acceptable salt thereof, wherein the compound has the following structure:

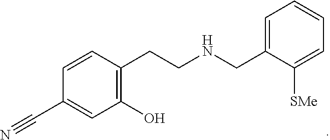 [SMILES: CSc1ccccc1CNCCc1ccc(C#N)cc1O]

8. The compound as claimed in claim 4, or isotopologue or pharmaceutically acceptable salt thereof, wherein: (i) X is CF₃; (ii) Z is CH₃; and (iii) R⁸ is OH.

9. The compound as claimed in claim 8, or isotopologue or pharmaceutically acceptable salt thereof, wherein R¹, R², R³, a, b, y, c, R⁴, R⁵, R⁶, and R⁷ are H.

10. The compound as claimed in claim 8, or isotopologue or pharmaceutically acceptable salt thereof, wherein the compound has the following structure:

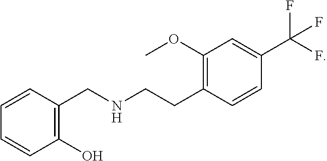 [SMILES: COc1cc(C(F)(F)F)ccc1CCNCc1ccccc1O]

11. The compound as claimed in claim 2, or isotopologue or pharmaceutically acceptable salt thereof, wherein R⁴ and c together form a heteroalkanediyl moiety.

12. The compound as claimed in claim 2, or isotopologue or pharmaceutically acceptable salt thereof, wherein R⁴ and R⁵ together form a heteroalkanediyl moiety.

13. The compound as claimed in claim 12, or isotopologue or pharmaceutically acceptable salt thereof, wherein the heteroalkanediyl moiety is selected from the group consisting of NHCH₂CH₂, NHCH₂NH, and SCH₂CH₂.

14. The compound as claimed in claim 2, or isotopologue or pharmaceutically acceptable salt thereof, wherein R⁵ is selected from the group consisting of OR⁹, and F, Cl.

15. (canceled)

16. (canceled)

17. (canceled)

18. The compound as claimed in claim 2, or isotopologue or pharmaceutically acceptable salt thereof, wherein R⁶ is selected from the group consisting of H, F, Cl, and OR⁹.

19. The compound as claimed in claim 2, or isotopologue or pharmaceutically acceptable salt thereof, wherein R⁸ is selected from the group consisting of H, F, Cl, OH, OR⁹, and SCH₃.

20. (canceled)

21. (canceled)

22. (canceled)

23. The compound as claimed in claim 2, or isotopologue or pharmaceutically acceptable salt thereof, wherein R⁸ and R⁷ together form a heteroalkanediyl moiety.

24. The compound as claimed in claim 23, or isotopologue or pharmaceutically acceptable salt thereof, wherein the heteroalkanediyl moiety is selected from the group consisting of NHCH₂CH₂, NHCH₂NH, and SCH₂CH₂.

25. The compound as claimed in claim 2, or isotopologue or pharmaceutically acceptable salt thereof, wherein R⁷ is selected from the group consisting of OR⁹, and F, Cl.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. A method comprising administering to a patient an effective amount of the compound as claimed in claim 1, for the treatment of one or more of: (i) neuropsychiatric, neurodegenerative, neuroinflammatory, and pain disorders; (ii) hypertension, hypotension, cardiovascular disease, stroke, and stroke recovery; (iii) gut motility; (iii) ureter contractions in animals; and (iv) compulsive disorders, anxiety disorders, fear disorders and aggressiveness in animals.

38. (canceled)

39. (canceled)

40. (canceled)

41. (canceled)

42. (canceled)
